SEQUENCE LISTING (29 sequences)

SEQ ID NO: 1 - protein, 352 aa, Saccharomyces cerevisiae
MASEKEIRRERFLNVFPKLVEELNASLLAYGMPKEACDWYAHSLNYNTPGGKLNRGLSVVDTYAILSNKTVEQLGQEEYEKVAILGWCIELLQAYFLVADDMMDKSITRRGQPCWYKVPEVGEIAIWDAFMLEAAIYKLLKSHFRNEKYYIDITELFHEVTFQTELGQLMDLITAPEDKVDLSKFSLKKHSFIVTFKTAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSK

SEQ ID NO: 2 - protein, 353 aa, Saccharomyces cerevisiae
MASEKEIRRERFLNVFPKLVEELNASLLAYGMPKEACDWYAHSLNYNTPGGKLNRGLSVVDTYAILSNKTVEQLGQEEYEKVAILGWCIELLQAYFLVADDMMDKSITRRGQPCWYKVPEVGEIAIWDAFMLEAAIYKLLKSHFRNEKYYIDITELFHEVTFQTELGQLMDLITAPEDKVDLSKFSLKKHSFIVTFKTAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSKL

SEQ ID NO: 3 - protein, 555 aa, Citrus X limon
MRRSANYQPSIWDHDFLQSLNSNYTDEAYKRRAEELRGKVKIAIKDVIEPLDQLELIDNLQRLGLAHRFETEIRNILNNIYNNNKDYNWRKENLYATSLEFRLLRQHGYPVSQEVFNGFKDDQGGFICDDFKGILSLHEASYYSLEGESIMEEAWQFTSKHLKEVMISKNMEEDVFVAEQAKRALELPLHWKVPMLEARWFIHIYERREDKNHLLLELAKMEFNTLQAIYQEELKEISGWWKDTGLGEKLSFARNRLVASFLWSMGIAFEPQFAYCRRVLTISIALITVIDDIYDVYGTLDELEIFTDAVERWDINYALKHLPGYMKMCFLALYNFVNEFAYYVLKQQDFDLLLSIKNAWLGLIQAYLVEAKWYHSKYTPKLEEYLENGLVSITGPLIITISYLSGTNPIIKKELEFLESNPDIVHWSSKIFRLQDDLGTSSDEIQRGDVPKSIQCYMHETGASEEVARQHIKDMMRQMWKKVNAYTADKDSPLTGTTTEFLLNLVRMSHFMYLHGDGHGVQNQETIDVGFTLLFQPIPLEDKHMAFTASPGTKG

SEQ ID NO: 4 - protein, 558 aa, Citrus X limon
MRRSANYQPSIWDHDFLQSLNSNYTDEAYKRRAEELRGKVKIAIKDVIEPLDQLELIDNLQRLGLAHRFETEIRNILNNIYNNNKDYNWRKENLYATSLEFRLLRQHGYPVSQEVFNGFKDDQGGFICDDFKGILSLHEASYYSLEGESIMEEAWQFTSKHLKEVMISKNMEEDVFVAEQAKRALELPLHWKVPMLEARWFIHIYERREDKNHLLLELAKMEFNTLQAIYQEELKEISGWWKDTGLGEKLSFARNRLVASFLWSMGIAFEPQFAYCRRVLTISIALITVIDDIYDVYGTLDELEIFTDAVERWDINYALKHLPGYMKMCFLALYNFVNEFAYYVLKQQDFDLLLSIKNAWLGLIQAYLVEAKWYHSKYTPKLEEYLENGLVSITGPLIITISYLSGTNPIIKKELEFLESNPDIVHWSSKIFRLQDDLGTSSDEIQRGDVPKSIQCYMHETGASEEVARQHIKDMMRQMWKKVNAYTADKDSPLTGTTTEFLLNLVRMSHFMYLHGDGHGVQNQETIDVGFTLLFQPIPLEDKHMAFTASPGTKGSKL

SEQ ID NO: 5 - protein, 582 aa, Solanum elaeagnifolium
MCSQHSTKPFSHSPNISTNLILSSDGSNPTRRSGNYDPTKWDYEYIQSANNHYTGEKYMKRFNELKAKIKKELMMVHEESQELDKLELIDNLERLGVSYHFKDEIMQILRSINDQSNIAATSADSLYYTALKFRILRQHGFYISQDILNDFKDEKGNFKQSLCEDIEGLLQLYEASFLSTDSETSSLLESANTFATSRLRKYLDNLNGDVNENWRVELVRHALELPSHCMLLRVETRWYINMYEKIPNADPLLLEFAKLDFNIVQATHQQELRNLSRWWKESWLAEKLSFSRDRIVESFLWIAGMMFEPQRNELSRTLLTKVTAIATIIDDIYDVYGTLDELEIFTQAVERMEVKAMDELPDYMKVCYLTLINNTNEVAYEVLKEQGINVTPYLTKAWTDLCKAYLQEARWYYSGYTPSMEEYMENAWVSVGSPVMVVNAFFLVTNPITKEALEHLFLNKYTEIIRWSAAIIRLTDDLATSSNEMERGDVSKSIQCYMKEKGATEEEARKHINFMIRDAWKRINTAQRDNPLFCEEFISCGMNIARTGQTIYQHGDGVGIQNYEIQNRIYKLFFDPIAVSIP

SEQ ID NO: 6 - protein, 582 aa, Solanum elaeagnifolium; identical to SEQ ID NO: 5

SEQ ID NO: 7 - protein, 578 aa, Pinus taeda
MSSTTSVSNEDGVPRRIAGHHSNLWDDDSIASLSTSYEAPSYRKRADKLIGEVKNIFDLMSVEDGVFTSPLSDLHHRLWMVDSVERLGIDRHFKDEINSALDHVYSYWTEKGIGRGRESGVTDLNSTALGLRTLRLHGYTVSSHVLDHFKNEKGQFTCSAIQTEGEIRDVLNLFRASLIAFPGEKIMEAAEIFSTMYLKDALQKIPPSGLSQEIEYLLEFGWHTNLPRMETRMYIDVFGEDTTFETPYLIREKLLELAKLEFNIFHSLVKRELQSLSRWWKDYGFPEITFSRHRHVEYYTLAACIANDPKHSAFRLGFGKISHMITILDDIYDTFGTMEELKLLTAAFKRWDPSSIECLPDYMKGVYMAVYDNINEMAREAQKIQGWDTVSYARKSWEAFIGAYIQEAKWISSGYLPTFDEYLENGKVSFGSRITTLEPMLTLGFPLPPRILQEIDFPSKFNDLICAILRLKGDTQCYKADRARGEEASAVSCYMKDHPGITEEDAVNQVNAMVDNLTKELNWELLRPDSGVPISYKKVAFDICRVFHYGYKYRDGFSVASIEIKNLVTRTVVETVPL

SEQ ID NO: 8 - protein, 581 aa, Pinus taeda
MSSTTSVSNEDGVPRRIAGHHSNLWDDDSIASLSTSYEAPSYRKRADKLIGEVKNIFDLMSVEDGVFTSPLSDLHHRLWMVDSVERLGIDRHFKDEINSALDHVYSYWTEKGIGRGRESGVTDLNSTALGLRTLRLHGYTVSSHVLDHFKNEKGQFTCSAIQTEGEIRDVLNLFRASLIAFPGEKIMEAAEIFSTMYLKDALQKIPPSGLSQEIEYLLEFGWHTNLPRMETRMYIDVFGEDTTFETPYLIREKLLELAKLEFNIFHSLVKRELQSLSRWWKDYGFPEITFSRHRHVEYYTLAACIANDPKHSAFRLGFGKISHMITILDDIYDTFGTMEELKLLTAAFKRWDPSSIECLPDYMKGVYMAVYDNINEMAREAQKIQGWDTVSYARKSWEAFIGAYIQEAKWISSGYLPTFDEYLENGKVSFGSRITTLEPMLTLGFPLPPRILQEIDFPSKFNDLICAILRLKGDTQCYKADRARGEEASAVSCYMKDHPGITEEDAVNQVNAMVDNLTKELNWELLRPDSGVPISYKKVAFDICRVFHYGYKYRDGFSVASIEIKNLVTRTVVETVPLSKL

SEQ ID NO: 9 - protein, 540 aa, Salvia pomifera
MRRSGDYQPSLWDFNYIQSLNTPYKEQRYVNRQAELIMQVRMLLKVKMEAIQQLELIDDLQYLGLSYFFPDEIKQILSSIHNEHRYFHNNDLYLTALGFRILRQHGFNVSEDVFDCFKTEKCSDFNANLAQDTKGMLQLYEASFLLREGEDTLELARRFSTRSLREKLDEDGDEIDEDLSSWIRHSLDLPLHWRIQGLEARWFLDAYARRPDMNPLIFKLAKLNFNIVQATYQEELKDVSRWWNSSCLAEKLPFVRDRIVECFFWAIGAFEPHQYSYQRKMAAIIITFVTIIDDVYDVYGTLEELELFTDMIRRWDNISISQLPYYMQVCYLALYNFVSERAYDILKDQHFNSIPYLQRSWVSLVEGYLKEAYWYYNGYKPSLEEYLNNAKISISAPTIISQLYFTLANSTDETVIESLYEYHNILYLSGTILRLADDLGTSQHELERGDVPKAIQCYMKDTNASEREAVEHVKFLIRETWKEMNTVTTASDCPFTDDLVAVATNLARAAQFIYLDGDGIGVQHSEIHQQMGGLLFQPYV

SEQ ID NO: 10 - protein, 543 aa, Salvia pomifera
MRRSGDYQPSLWDFNYIQSLNTPYKEQRYVNRQAELIMQVRMLLKVKMEAIQQLELIDDLQYLGLSYFFPDEIKQILSSIHNEHRYFHNNDLYLTALGFRILRQHGFNVSEDVFDCFKTEKCSDFNANLAQDTKGMLQLYEASFLLREGEDTLELARRFSTRSLREKLDEDGDEIDEDLSSWIRHSLDLPLHWRIQGLEARWFLDAYARRPDMNPLIFKLAKLNFNIVQATYQEELKDVSRWWNSSCLAEKLPFVRDRIVECFFWAIGAFEPHQYSYQRKMAAIIITFVTIIDDVYDVYGTLEELELFTDMIRRWDNISISQLPYYMQVCYLALYNFVSERAYDILKDQHFNSIPYLQRSWVSLVEGYLKEAYWYYNGYKPSLEEYLNNAKISISAPTIISQLYFTLANSTDETVIESLYEYHNILYLSGTILRLADDLGTSQHELERGDVPKAIQCYMKDTNASEREAVEHVKFLIRETWKEMNTVTTASDCPFTDDLVAVATNLARAAQFIYLDGDGIGVQHSEIHQQMGGLLFQPYVSKL

SEQ ID NO: 11 - protein, 534 aa, Ocimum basilicum
MSACTPLASAMPLSSTPLINGDNSQRKNTRQHMEESSSKRREYLLEETTRKLQRNDTESVEKLKLIDNIQQLGIGYYFEDAINAVLRSPFSTGEEDLFTAALRFRLLRHNGIEISPEIFLKFKDERGKFDESDTLGLLSLYEASNLGVAGEEILEEAMEFAEARLRRSLSEPAAPLHGEVAQALDVPRHLRMARLEARRFIEQYGKQSDHDGDLLELAILDYNQVQAQHQSELTEIIRWWKELGLVDKLSFGRDRPLECFLWTVGLLPEPKYSSVRIELAKAISILLVIDDIFDTYGEMDDLILFTDAIRRWDLEAMEGLPEYMKICYMALYNTTNEVCYKVLRDTGRIVLLNLKSTWIDMIEGFMEEAKWFNGGSAPKLEEYIENGVSTAGAYMAFAHIFFLIGEGVTHQNSQLFTQKPYPKVFSAAGRILRLWDDLGTAKEEQERGDLASCVQLFMKEKSLTEEEARSRILEEIKGLWRDLNGELVYNKNLPLSIIKVALNMARASQVVYKHDQDTYFSSVDNYVDALFFTQ

SEQ ID NO: 12 - protein, 537 aa, Ocimum basilicum
MSACTPLASAMPLSSTPLINGDNSQRKNTRQHMEESSSKRREYLLEETTRKLQRNDTESVEKLKLIDNIQQLGIGYYFEDAINAVLRSPFSTGEEDLFTAALRFRLLRHNGIEISPEIFLKFKDERGKFDESDTLGLLSLYEASNLGVAGEEILEEAMEFAEARLRRSLSEPAAPLHGEVAQALDVPRHLRMARLEARRFIEQYGKQSDHDGDLLELAILDYNQVQAQHQSELTEIIRWWKELGLVDKLSFGRDRPLECFLWTVGLLPEPKYSSVRIELAKAISILLVIDDIFDTYGEMDDLILFTDAIRRWDLEAMEGLPEYMKICYMALYNTTNEVCYKVLRDTGRIVLLNLKSTWIDMIEGFMEEAKWFNGGSAPKLEEYIENGVSTAGAYMAFAHIFFLIGEGVTHQNSQLFTQKPYPKVFSAAGRILRLWDDLGTAKEEQERGDLASCVQLFMKEKSLTEEEARSRILEEIKGLWRDLNGELVYNKNLPLSIIKVALNMARASQVVYKHDQDTYFSSVDNYVDALFFTQSKL

SEQ ID NO: 13 - protein, 323 aa, Cannabis sativa
MSAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 14 - protein, 326 aa, Cannabis sativa
MSAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFISKL

SEQ ID NO: 15 - protein, 920 aa, synthetic construct
  REGION 1..920: note = the fusion protein between having a GPP synthase domain (Erg20pN127W) and a terpene synthase domain (ClLimS) linked by a 5xGS polypeptide and targeted to the peroxisome by a C-terminal PTS1
MASEKEIRRERFLNVFPKLVEELNASLLAYGMPKEACDWYAHSLNYNTPGGKLNRGLSVVDTYAILSNKTVEQLGQEEYEKVAILGWCIELLQAYFLVADDMMDKSITRRGQPCWYKVPEVGEIAIWDAFMLEAAIYKLLKSHFRNEKYYIDITELFHEVTFQTELGQLMDLITAPEDKVDLSKFSLKKHSFIVTFKTAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSKGSGSGSGSGSMRRSANYQPSIWDHDFLQSLNSNYTDEAYKRRAEELRGKVKIAIKDVIEPLDQLELIDNLQRLGLAHRFETEIRNILNNIYNNNKDYNWRKENLYATSLEFRLLRQHGYPVSQEVFNGFKDDQGGFICDDFKGILSLHEASYYSLEGESIMEEAWQFTSKHLKEVMISKNMEEDVFVAEQAKRALELPLHWKVPMLEARWFIHIYERREDKNHLLLELAKMEFNTLQAIYQEELKEISGWWKDTGLGEKLSFARNRLVASFLWSMGIAFEPQFAYCRRVLTISIALITVIDDIYDVYGTLDELEIFTDAVERWDINYALKHLPGYMKMCFLALYNFVNEFAYYVLKQQDFDLLLSIKNAWLGLIQAYLVEAKWYHSKYTPKLEEYLENGLVSITGPLIITISYLSGTNPIIKKELEFLESNPDIVHWSSKIFRLQDDLGTSSDEIQRGDVPKSIQCYMHETGASEEVARQHIKDMMRQMWKKVNAYTADKDSPLTGTTTEFLLNLVRMSHFMYLHGDGHGVQNQETIDVGFTLLFQPIPLEDKHMAFTASPGTKGSKL

SEQ ID NO: 16 - protein, 919 aa, synthetic construct
  REGION 1..919: note = the fusion protein between having a terpene synthase domain (ClLimS) and a GPP synthase domain (Erg20pN127W) linked by a 5xGS polypeptide and targeted to the peroxisome by a C-terminal PTS1
MRRSANYQPSIWDHDFLQSLNSNYTDEAYKRRAEELRGKVKIAIKDVIEPLDQLELIDNLQRLGLAHRFETEIRNILNNIYNNNKDYNWRKENLYATSLEFRLLRQHGYPVSQEVFNGFKDDQGGFICDDFKGILSLHEASYYSLEGESIMEEAWQFTSKHLKEVMISKNMEEDVFVAEQAKRALELPLHWKVPMLEARWFIHIYERREDKNHLLLELAKMEFNTLQAIYQEELKEISGWWKDTGLGEKLSFARNRLVASFLWSMGIAFEPQFAYCRRVLTISIALITVIDDIYDVYGTLDELEIFTDAVERWDINYALKHLPGYMKMCFLALYNFVNEFAYYVLKQQDFDLLLSIKNAWLGLIQAYLVEAKWYHSKYTPKLEEYLENGLVSITGPLIITISYLSGTNPIIKKELEFLESNPDIVHWSSKIFRLQDDLGTSSDEIQRGDVPKSIQCYMHETGASEEVARQHIKDMMRQMWKKVNAYTADKDSPLTGTTTEFLLNLVRMSHFMYLHGDGHGVQNQETIDVGFTLLFQPIPLEDKHMAFTASPGTKGGSGSGSGSGSKMASEKEIRRERFLNVFPKLVEELNASLLAYGMPKEACDWYAHSLNYNTPGGKLNRGLSVVDTYAILSNKTVEQLGQEEYEKVAILGWCIELLQAYFLVADDMMDKSITRRGQPCWYKVPEVGEIAIWDAFMLEAAIYKLLKSHFRNEKYYIDITELFHEVTFQTELGQLMDLITAPEDKVDLSKFSLKKHSFIVTFKTAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSKL

SEQ ID NO: 17 - protein, 493 aa, Catharanthus roseus
MDYLTIILTLLFALTLYEAFSYLSRRTKNLPPGPSPLPFIGSLHLLGDQPHKSLAKLSKKHGPIMSLKLGQITTIVISSSTMAKEVLQKQDLAFSSRSVPNALHAHNQFKFSVVWLPVASRWRSLRKVLNSNIFSGNRLDANQHLRTRKVQELIAYCRKNSQSGEAVDVGRAAFRTSLNLLSNLIFSKDLTDPYSDSAKEFKDLVWNIMVEAGKPNLVDFFPLLEKVDPQGIRHRMTIHFGEVLKLFGGLVNERLEQRRSKGEKNDVLDVLLTTSQESPEEIDRTHIERMCLDLFVAGTDTTSSTLEWAMSEMLKNPDKMKKTQDELAQVIGRGKTIEESDINRLPYLRCVMKETLRIHPPVPFLIPRKVEQSVEVCGYNVPKGSQVLVNAWAIGRDETVWDDALAFKPERFMESELDIRGRDFELIPFGAGRRICPGLPLALRTVPLMLGSLLNSFNWKLEGGMAPKDLDMEEKFGITLQKAHPLRAVPSTL

SEQ ID NO: 18 - protein, 714 aa, Catharanthus roseus
MDSSSEKLSPFELMSAILKGAKLDGSNSSDSGVAVSPAVMAMLLENKELVMILTTSVAVLIGCVVVLIWRRSSGSGKKVVEPPKLIVPKSVVEPEEIDEGKKKFTIFFGTQTGTAEGFAKALAEEAKARYEKAVIKVIDIDDYAADDEEYEEKFRKETLAFFILATYGDGEPTDNAARFYKWFVEGNDRGDWLKNLQYGVFGLGNRQYEHFNKIAKVVDEKVAEQGGKRIVPLVLGDDDQCIEDDFAAWRENVWPELDNLLRDEDDTTVSTTYTAAIPEYRVVFPDKSDSLISEANGHANGYANGNTVYDAQHPCRSNVAVRKELHTPASDRSCTHLDFDIAGTGLSYGTGDHVGVYCDNLSETVEEAERLLNLPPETYFSLHADKEDGTPLAGSSLPPPFPPCTLRTALTRYADLLNTPKKSALLALAAYASDPNEADRLKYLASPAGKDEYAQSLVANQRSLLEVMAEFPSAKPPLGVFFAAIAPRLQPRFYSISSSPRMAPSRIHVTCALVYEKTPGGRIHKGVCSTWMKNAIPLEESRDCSWAPIFVRQSNFKLPADPKVPVIMIGPGTGLAPFRGFLQERLALKEEGAELGTAVFFFGCRNRKMDYIYEDELNHFLEIGALSELLVAFSREGPTKQYVQHKMAEKASDIWRMISDGAYVYVCGDAKGMARDVHRTLHTIAQEQGSMDSTQAEGFVKNLQMTGRYLRDVW

SEQ ID NO: 19 - protein, 323 aa, Aspergillus terreus
MSLVSGPGRATGAHADQELCVQKHVEYIKNLDSRRDELEYWLTEHLRLNGVYWGLTALCLLGHPEALPREDTINFVLSCQRENGGFAAAPGHDAHMLYTVSAVQILVTLDAVDELEKRGLGGKEKVGSFIAGLQDKDTGSFMGDEWGELDTRFLYGAFNALSLLGLLDTIDVPKAVSYIQKCENLDGAYGIRPGAESHAGQVFTCVAALAIAGRLDLVNKDRLGGWLSERQLENGGFNGRPEKLEDACYSWWVGSSLAMIDKLHWIDGKKLASFILRCQDPEAGGFSDRPGNMVDVFHTHFAIAGLSLLNKFTSRERAQVDEC

SEQ ID NO: 20 - protein, 472 aa, Neosartorya fumigata
MSIGAEIDSLVPAPPGLNGTAAGYPAKTQKELSNGDFDAHDGLSLAQLTPYDVLTAALPLPAPASSTGFWWRETGPVMSKLLAKANYPLYTHYKYLMLYHTHILPLLGPRPPLENSTHPSPSNAPWRSFLTDDFTPLEPSWNVNGNSEAQSTIRLGIEPIGFEAGAAADPFNQAAVTQFMHSYEATEVGATLTLFEHFRNDMFVGPETYAALRAKIPEGEHTTQSFLAFDLDAGRVTTKAYFFPILMSLKTGQSTTKVVSDSILHLALKSEVWGVQTIAAMSVMEAWIGSYGGAAKTEMISVDCVNEADSRIKIYVRMPHTSLRKVKEAYCLGGRLTDENTKEGLKLLDELWRTVFGIDDEDAELPQNSHRTAGTIFNFELRPGKWFPEPKVYLPVRHYCESDMQIASRLQTFFGRLGWHNMEKDYCKHLEDLFPHHPLSSSTGTHTFLSFSYKKQKGVYMTMYYNLRVYST

SEQ ID NO: 21 - protein, 401 aa, Artemisia capillaris
MASLTVGSLCKPTSNGLSILVTSSSSLSTGAHASNFLRISKVENNVSAQFQRRGYKNHFGQSLHEPLSLQKMDEEKFKLNAASTNNPQFDATHDLVKPTESVISFLEVLFRFIRPYAAVGTVLCIASMSLLTVEKLSDFSPLFFMKVLQALVGAMFMQMWVCGINQICDIELDKINKPSLPLASGELSMTTAITVSALSAIMSFSIGWIASPALFWGFVGWFVVGTAYSANLPWLRWKRFPLTSAFYMLCSRALVVPIGYYLHMQKSIHGGSALLSRPILFAVGMLSAFCISTIFFKDIPDIEGDRMHGIKSLAITLGEKRTFWMCIWILEIAYVAAAFFGATSPITWSKYITVISHLAMALALWTRAKSTDVKNKDAVQSMYYFLWQLFFAEYGLIALVR

SEQ ID NO: 22 - protein, 541 aa, Mentha citrata
MATRRSGNYHPSVWDFDFIQSLDTDHYKEEKQLEREEELIMEVKKLLGAKMEATKQLELIDDLQNLGLSYFFRDEIKNILNSIYKIFQNNNSTKVGDLHFTSLGFRLLRQHGFNVSQGVFDCFKNEHGSDFEKTLIGEDTKGVLQLYEASFLLREGEDTLEVARKFSTEFLEEKLKAGIDGDNLSSSIGHSLEIPLHWRIQRLEERWFLDAYSRRKDMNPIIFELAKLDFNIIQATQQEELKDLSRWWNDSSLPQKLPFVRDRLVESYYWALGLFEAHKFGYERKTAAKIITLITALDDVYDIYGTLDELQLFTHVIRRWDTESATQLPYYLQLFYFVLYNFVSEVAYHILKEEGFISIPFLHRAWVDLVEGYLQEAKWYYTKYTPTMEEYLNYASITIGAPAVISQIYFMLAKSKEKPVIESFYEYDEIIRLSGMLVRLPDDLGTLPFEMKRGDVAKSIQIYMKEQNATREEAEEHVRFMIREAWKEMNTTMAANSDLRGDVVMAAANLGRDAQFMYLDGDGNHSQLQHRIANLLFKPYV

SEQ ID NO: 23 - protein, 544 aa, Mentha citrata
MATRRSGNYHPSVWDFDFIQSLDTDHYKEEKQLEREEELIMEVKKLLGAKMEATKQLELIDDLQNLGLSYFFRDEIKNILNSIYKIFQNNNSTKVGDLHFTSLGFRLLRQHGFNVSQGVFDCFKNEHGSDFEKTLIGEDTKGVLQLYEASFLLREGEDTLEVARKFSTEFLEEKLKAGIDGDNLSSSIGHSLEIPLHWRIQRLEERWFLDAYSRRKDMNPIIFELAKLDFNIIQATQQEELKDLSRWWNDSSLPQKLPFVRDRLVESYYWALGLFEAHKFGYERKTAAKIITLITALDDVYDIYGTLDELQLFTHVIRRWDTESATQLPYYLQLFYFVLYNFVSEVAYHILKEEGFISIPFLHRAWVDLVEGYLQEAKWYYTKYTPTMEEYLNYASITIGAPAVISQIYFMLAKSKEKPVIESFYEYDEIIRLSGMLVRLPDDLGTLPFEMKRGDVAKSIQIYMKEQNATREEAEEHVRFMIREAWKEMNTTMAANSDLRGDVVMAAANLGRDAQFMYLDGDGNHSQLQHRIANLLFKPYVSKL

SEQ ID NO: 24 - protein, 544 aa, Mentha spicata
MERRSGNYNPSRWDVNFIQSLLSDYKEDKHVIRASELVTLVKMELEKETDQIRQLELIDDLQRMGLSDHFQNEFKEILSSIYLDHHYYKNPFPKEERDLYSTSLAFRLLREHGFQVAQEVFDSFKNEEGEFKESLSDDTRGLLQLYEASFLLTEGETTLESAREFATKFLEEKVNEGGVDGDLLTRIAYSLDIPLHWRIKRPNAPVWIEWYRKRPDMNPVVLELAILDLNIVQAQFQEELKESFRWWRNTGFVEKLPFARDRLVECYFWNTGIIEPRQHASARIMMGKVNALITVIDDIYDVYGTLEELEQFTDLIRRWDINSIDQLPDYMQLCFLALNNFVDDTSYDVMKEKGVNVIPYLRQSWVDLADKYMVEARWFYGGHKPSLEEYLENSWQSISGPCMLTHIFFRVTDSFTKETVDSLYKYHDLVRWSSFVLRLADDLGTSVEEVSRGDVPKSLQCYMSDYNASEAEARKHVKWLIAEVWKKMNAERVSKDSPFGKDFIGCAVDLGRMAQLMYHNGDGHGTQHPIIHQQMTRTLFEPFA

SEQ ID NO: 25 - protein, 547 aa, Mentha spicata
MERRSGNYNPSRWDVNFIQSLLSDYKEDKHVIRASELVTLVKMELEKETDQIRQLELIDDLQRMGLSDHFQNEFKEILSSIYLDHHYYKNPFPKEERDLYSTSLAFRLLREHGFQVAQEVFDSFKNEEGEFKESLSDDTRGLLQLYEASFLLTEGETTLESAREFATKFLEEKVNEGGVDGDLLTRIAYSLDIPLHWRIKRPNAPVWIEWYRKRPDMNPVVLELAILDLNIVQAQFQEELKESFRWWRNTGFVEKLPFARDRLVECYFWNTGIIEPRQHASARIMMGKVNALITVIDDIYDVYGTLEELEQFTDLIRRWDINSIDQLPDYMQLCFLALNNFVDDTSYDVMKEKGVNVIPYLRQSWVDLADKYMVEARWFYGGHKPSLEEYLENSWQSISGPCMLTHIFFRVTDSFTKETVDSLYKYHDLVRWSSFVLRLADDLGTSVEEVSRGDVPKSLQCYMSDYNASEAEARKHVKWLIAEVWKKMNAERVSKDSPFGKDFIGCAVDLGRMAQLMYHNGDGHGTQHPIIHQQMTRTLFEPFASKL

SEQ ID NO: 26 - protein, 569 aa, Ocimum basilicum
MSSTRRRPSCPLQLDVEPRRSGNYQPSAWDFNYIQSLNNNHSKEERHLERKAKLIEEVKMLLEQEMAAVQQLELIEDLKNLGLSYLFQDEIKIILNSIYNHHKCFHNNHEQCIHVNSDLYFVALGFRLFRQHGFKVSQEVFDCFKNEEGSDFSANLADDTKGLLQLYEASYLVTEDEDTLEMARQFSTKILQKKVEEKMIEKENLLSWTLHSLELPLHWRIQRLEAKWFLDAYASRPDMNPIIFELAKLEFNIAQALQQEELKDLSRWWNDTGIAEKLPFARDRIVESHYWAIGTLEPYQYRYQRSLIAKIIALTTVVDDVYDVYGTLDELQLFTDAIRRWDIESINQLPSYMQLCYLAIYNFVSELAYDIFRDKGFNSLPYLHKSWLDLVEAYFVEAKWFHDGYTPTLEEYLNNSKITIICPAIVSEIYFAFANSIDKTEVESIYKYHDILYLSGMLARLPDDLGTSSFEMKRGDVAKAIQCYMKEHNASEEEAREHIRFLMREAWKHMNTAAAADDCPFESDLVVGAASLGRVANFVYVEGDGFGVQHSKIHQQMAELLFYPYQSKL

SEQ ID NO: 27 - protein, 496 aa, Mentha spicata
MELQISSAIIILVATFVASLLIKQWRKSESRQNLPPGPPKLPLVGHLHLLWGKLPQHAMADMAKKYGPVTHVQLGEVFSVVLSSREATKEAMKLLDPACADRFESIGTRIMWYDNDDIIFSPYSDHWRQMRKICVSELLSARNVRSFGFIRQDEMSRLLRHLQSSAGETVDMTERIATLTCSIICRAAFGAIINDHEELVELVKDSLSMASGFELADLFPSSKLLNLLCWNKSKLWRMRRRVDTILEAIVDEHKLKKSGEFGGEDIIDVLFRMQKDSQIKVPITTNAIKAFIFDTFSAGTETSSTTTLWVMAELMRNPAVMAKAQAEVRAALKGKTSVDVDDVQELKYMKSVVKETMRMHPPIPLIPRSCREECEVNGYKIPNKARIMINVWSMGRNPLYWEKPETFWPERFDQVSRDFMGSDFEFIPFGAGRRICPGLNFGLANVEVPLAQLLYHFDWKLAEGMKPSDMDMSEAEGLTGIRKNNLLLVPTLYKSP

SEQ ID NO: 28 - protein, 717 aa, Taxus cuspidata
MQANSNTVEGASQGKSLLDISRLDHIFALLLNGKGGDLGAMTGSALILTENSQNLMILTTALAVLVACVFFFVWRRGGSDTQKPAVRPTPLVKEEDEEEEDDSAKKKVTIFFGTQTGTAEGFAKALAEEAKARYEKAVFKVVDLDNYAADDEQYEEKLKKEKLAFFMLATYGDGEPTDNAARFYKWFLEGKEREPWLSDLTYGVFGLGNRQYEHFNKVAKAVDEVLIEQGAKRLVPVGLGDDDQCIEDDFTAWREQVWPELDQLLRDEDDEPTSATPYTAAIPEYRVEIYDSVVSVYEETHALKQNGQAVYDIHHPCRSNVAVRRELHTPLSDRSCIHLEFDISDTGLIYETGDHVGVHTENSIETVEEAAKLLGYQLDTIFSVHGDKEDGTPLGGSSLPPPFPGPCTLRTALARYADLLNPPRKAAFLALAAHASDPAEAERLKFLSSPAGKDEYSQWVTASQRSLLEIMAEFPSAKPPLGVFFAAIAPRLQPRYYSISSSPRFAPSRIHVTCALVYGPSPTGRIHKGVCSNWMKNSLPSEETHDCSWAPVFVRQSNFKLPADSTTPIVMVGPGTGFAPFRGFLQERAKLQEAGEKLGPAVLFFGCRNRQMDYIYEDELKGYVEKGILTNLIVAFSREGATKEYVQHKMLEKASDTWSLIAQGGYLYVCGDAKGMARDVHRTLHTIVQEQESVDSSKAEFLVKKLQMDGRYLRDIW

SEQ ID NO: 29 - protein, 334 aa, Cannabis sativa
MSQRLQSIKDHLSAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

(19) United States
(12) Patent Application Publication
(10) Pub. No.: US 2026/0234674 A1
(43) Pub. Date: Aug. 13, 2026

(54) Production of Geranyl Diphosphate-Derived Compounds

(71) Applicant: Københavns Universitet, København K (DK)

(72) Inventors: Sotirios KAMPRANIS, Hellerup (DK); Simon DUSSÉAUX, København (DK); Codruta IGNEA, Montreal (CA); William Thomas WAJN, Frederiksberg (DK)

(21) Appl. No.: 19/430,857

(22) Filed: Dec. 23, 2025

Related U.S. Application Data
(63) Continuation of application No. 17/917,244, filed on Apr. 7, 2021, now Pat. No. 12,529,077.

(30) Foreign Application Priority Data
Apr. 8, 2020 (DK) PA 2020 70216

(51) Int. Cl. C12P 5/00; C12N 9/10; C12N 9/88

(52) U.S. Cl. CPC C12P 5/005; C12N 9/1085; C12N 9/88; C12P 5/007; C12Y 205/01029; C12Y 402/03

(57) ABSTRACT

Disclosed is yeast cells having peroxisomally localized GPP synthase and a peroxisomally localized enzyme that converts GPP into a monoterpenoids, cannabinoids, monoterpene indole alkaloids and prenylated aromatic compounds; or a precursor therefore, which yeast cells are capable of producing improved amounts of monoterpenoids, cannabinoids, monoterpene indole alkaloids and prenylated aromatic compounds, compared with the same yeast cells where the GPP synthase and the enzyme that converts GPP are located in the cytoplasm. Further disclosed is the use of the yeast cell for producing monoterpenoids, cannabinoids, monoterpene indole alkaloids and prenylated aromatic compounds.

### REFERENCE TO SEQUENCE LISTING

The instant application contains a Sequence Listing that has been submitted electronically and is hereby incorporated by reference in its entirety. The Sequence Listing was created on Dec. 22, 2025, is named “25-0836-WO—US—CON_SequenceListing.xml”, and is 42,501 bytes in size.

### FIELD OF THE INVENTION

The present invention relates to the production of monoterpenoids, cannabinoids, iridoids, monoterpene indole alkaloids, and prenylated aromatic compounds in eukaryotic cells, such as yeast cells. The invention further relates to engineered yeast cells, particularly adapted to such production.

### BACKGROUND OF THE INVENTION

Terpenes, terpenoids, derivatives thereof and other prenylated aromatic compounds are widely used e.g. as pharmaceuticals, cosmetics, nutraceuticals, flavors, fragrances and pesticides. Methods for increasing the production of these compounds in natural or engineered cells are abundant in the art.

Using engineered microorganisms for producing valuable molecules from renewable feedstock is a desirable alternative from conventional means of production. However, achieving economically viable yield, titers and productivity is a major roadblock towards industrialization. Obstacles often encountered arise from the standoff between the engineered pathway and the native metabolism that are pulling in opposite directions. Metabolism has evolved towards meeting the needs for growth and rerouting it can be challenging due to multiple layers of control, such as gene regulation, negative feedback loops at the enzyme level by downstream products, and efficient competing pathways.

Monoterpenes and other geranyl diphosphate (GPP)-derived compounds, which are widely used as flavors, fragrances, pesticides and could find applications as drop in jet fuel or biopolymers, are a prime example of these issues. On one hand, extraction from plant natural sources can hardly meet the increasing demands and represents an environmental challenge, whereas, on the other hand, production by microbial host leads to low yield and is hindered by native metabolism constraints.

Monoterpene production by engineered microbes relies on either the MEP pathway (mainly prokaryotes), the MVA pathway, or the alternative MVA pathway, all three leading to the formation of DMAPP and IPP, which are, in turn, condensed to form GPP. GPP is converted either into a wide array of monoterpenes by monoterpene synthases (MTSs) that rearrange the 10-carbons backbone of GPP into various monoterpenes or precursors thereof, or it is further elongated into FPP or GGPP by successive addition of IPP molecules to form sesquiterpenes and diterpenes respectively. GPP also serves as the precursor for the synthesis of a number of compounds that contain a terpene moiety, such as cannabinoids, iridoids, monoterpene indole alkaloids, prenylated aromatic compounds, and other meroterpenoids.

Yeast is considered a good host for terpene production because of its ease to be engineered, its native mevalonate pathway, and a good capacity to harbor functional cytochromes P450 in its endoplasmic reticulum (ER) membrane for terpene scaffold decoration. It has shown great capacity at producing sesquiterpenes, such as artemisinin and farnesene, at industrial scale. However, the production of monoterpenes has so far been far less successful.

This can be mainly explained by the rope-pulling game that is played at the GPP branch-point between native sterol biosynthesis and the heterologous pathway leading to monoterpenes, and which is largely in favor of the native metabolism. In wild-type yeast, there are no GPP-based compounds produced and the only purpose of GPP is to serve as an intermediate that is further elongated into FPP for the production of squalene in the sterol pathway. Because of this, no dedicated GPP synthase is present in yeast, and GPP is produced by a bi-functional GPP-FPP synthase, Erg20p, that has been shown to convert very efficiently GPP into FPP as soon as it is formed and channel it into sterol synthesis. Various strategies have been employed to downregulate Erg20p, either by converting it into a strict GPP synthase, or by reducing its activity, but the intrinsic essentiality of sterol synthesis have rendered those attempts only moderate fruitful, while decreasing cells viability by posing a burden on sterol synthesis. Compartmentalization is a strategy used by eukaryotic cells to solve similar issues within their own metabolism. Organelles, such as mitochondria, peroxisomes, and the endoplasmic reticulum (ER), are designed to protect the rest of the cells from toxic compounds, isolate intermediates from competing pathways, shield enzymes from inhibitors, and, overall, provide a more suitable environment for a reaction to occur away from the main bulk of the metabolism.

An example of such a strategy has been reported recently, where geraniol production has been improved 11.5-fold by compartmentalizing an extra copy of the entire MVA pathway into the mitochondria together with a geraniol synthase, in comparison with the same modification in the cytosol.

While this proved to be a successful strategy, hijacking the mitochondria appeared to pose a metabolic burden to the strain with lower cells viability and growth. This can be attributed to the essential nature of the mitochondria as the powerhouse of the cells, which may hinder further engineering to reach the significantly higher titer needed for industrial application. These findings also showed that mitochondria might come with limitations on how far they can be engineered without compromising the integrity of the metabolism.

US20150010978 discloses methods for producing terpenoids in a vast number of cells by transforming the cells with genes encoding enzymes involved in the biosynthesis of the terpenoids. The genes may be introduced into the genomes of chloroplasts for cells having chloroplasts. The exemplification discloses production of di-terpenes.

KR20190079575A discloses a recombinant yeast wherein the number of peroxisomes is increased, leading to increased terpenoid production. Also disclosed is insertion of a heterologous geranylgeranyl pyrophosphate synthase.

US20130302861A1 discloses terpenoid production in yeast by localizing a terpene synthase to the mitochondria. The exemplification focuses on FPP-derived sesquiterpenes.

Guo-Song Liu et al (J. Agric. Food Chem. 2020, 68, 7, 2132-2138) reported the production of squalene, the FPP-based precursor of ergosterol, in yeast peroxisome demonstrating the functionality of the MVA pathway in this organelle. However, the resulting strain did not outperform its cytosolic counterpart, most probably due to the fact that the original pathway is already well-tuned and designed to efficiently produce squalene in the cytosol.

### SUMMARY OF THE INVENTION

In a first aspect the invention relates to a yeast cell comprising a peroxisomally-localized enzyme catalyzing the formation of the branch point compound, which branch point compound can be converted in a prioritized pathway and in a non-prioritized pathway; and a peroxisomally-localized enzyme catalyzing the first step of the non-prioritized pathway. In a preferred embodiment, the invention relates to a yeast cell comprising a peroxisomally-localized GPP synthase and a peroxisomally-localized monoterpene synthase.

In a second aspect, the invention relates to a method for producing monoterpenoids, cannabinoids, iridoids, monoterpene indole alkaloids, and prenylated aromatic compounds using a yeast cell of the invention.

### DESCRIPTION OF THE DRAWINGS

FIG. 1 shows a chart disclosing the limonene production in yeast, where the limonene synthase (MTS) and/or the GPP synthase were localized to either the cytoplasm or the peroxisomes. For further details, see example 1.

FIG. 2 shows a chart disclosing the effect of localizing genes of the MVA pathway together with the limonene synthase (MTS) and the GPP synthase to the peroxisomes. For further details, see example 2.

FIG. 3 shows a chart disclosing improved production of six monoterpenoids, camphene, sabinene, (S)-(−)-limonene, alpha-pinene, (R)-(+)-limonene and (R)-(+)-linalool, by peroxisomal localization of the respective synthases. For further details, see example 3.

FIG. 4 shows graphs of the titer development in a fermentation of yeast according to the invention. FIG. 4A shows the production of (R)-(+)-limonene and FIG. 4B shows the production of geraniol. For further details, see example 4.

FIG. 5 shows a chart disclosing the effect of localizing a bi-functional GPP synthase/terpene synthase enzyme (GPP synthase-terpene synthase fusion) to the peroxisome on terpene production. For further details, see example 5.

FIG. 6 shows a graph disclosing the production of trans-isopiperitenol and 8-hydroxygeraniol. FIG. 6A shows (−)-limonene and trans-isopiperitenol production in strains CYTLim06, PERLim29 and PERLim30. FIG. 6B shows geraniol and 8-hydroxy-geraniol production in strains PERMGer03 and PERGer04.

FIG. 7 shows a graph disclosing the production of cannabinoid precursors. FIG. 7A shows CBGA production in strains PERMva01 and PERCan01 in culture supplemented with 0.5 mM OA. FIG. 7B shows CBGA production at different concentrations of OA added in the culture. FIG. 7C shows improved peroxisomal CBGA production by targeting CsPT4 to the peroxisome using an N-terminal targeting signal in strain PERCan02.

### OVERVIEW OF SEQUENCE LISTING

SEQ ID NO: 1 is the amino acid sequence of the engineered geranyl diphosphate synthase derived from the Saccharomyces cerevisiae Erg20p protein and indicated as Erg20p^{N127W}.

SEQ ID NO: 2: is the amino acid sequence of the geranyl diphosphate synthase derived from the Saccharomyces cerevisiae Erg20p protein and indicated as Erg20p^{N127W}, and provided with the SKL peroxisomal localization signal.

SEQ ID NO: 3: is the amino acid sequence of the (+)-limonene synthase derived from Citrus limon and encoded by the C/LimS gene.

SEQ ID NO: 4: is the amino acid sequence of the (+)-limonene synthase derived from Citrus limon and encoded by the C/LimS gene and provided with the SKL peroxisomal localization signal.

SEQ ID NO: 5: is the amino acid sequence of the camphene synthase derived from Solanum elaeagnifolium and encoded by the SeCamS gene.

SEQ ID NO: 6: is the amino acid sequence of the camphene synthase derived from Solanum elaeagnifolium and encoded by the SeCamS gene and provided with the SKL peroxisomal localization signal.

SEQ ID NO: 7: is the amino acid sequence of the α-pinene synthase derived from Pinus taeda and encoded by the PtPinS gene.

SEQ ID NO: 8: is the amino acid sequence of the α-pinene synthase derived from Pinus taeda and encoded by the PtPinS gene and provided with the SKL peroxisomal localization signal.

SEQ ID NO: 9: is the amino acid sequence of the sabinene synthase derived from Salvia pomifera and encoded by the SpSabS gene.

SEQ ID NO: 10: is the amino acid sequence of the sabinene synthase derived from Salvia pomifera and encoded by the SpSabS gene and provided with an SKL peroxisomal localization signal.

SEQ ID NO: 11: is the amino acid sequence of the geraniol synthase derived from Ocimum basilicum and encoded by the tObGES gene.

SEQ ID NO: 12: is the amino acid sequence of the geraniol synthase derived from Ocimum basilicum and encoded by the tObGES gene, and provided with the SKL peroxisomal localization signal.

SEQ ID NO: 13: is the amino acid sequence of the geranyldiphosphate:olivetolate geranyltransferase derived from Cannabis sativa and encoded by the CsPT4 gene. SEQ ID NO: 14: is the amino acid sequence of the geranyldiphosphate:olivetolate geranyltransferase derived from Cannabis sativa and encoded by the CsPT4 gene, and provided with the SKL peroxisomal localization signal.

SEQ ID NO: 15: is the amino acid sequence of the fusion protein between having a GPP synthase domain (Erg20p^{N127W}) and a terpene synthase domain (C/LimS) linked by a 5×GS polypeptide and targeted to the peroxisome by a C-terminal PTS1

SEQ ID NO: 16: is the amino acid sequence of the fusion protein between having a terpene synthase domain (C/LimS) and a GPP synthase domain (Erg20p^{N127W}) linked by a 5×GS polypeptide and targeted to the peroxisome by a C-terminal PTS1

SEQ ID NO: 17: is the amino acid sequence of the geraniol 8-hydroxylase derived from Catharanthous roseus and encoded by the CrG8OH gene.

SEQ ID NO: 18: is the amino acid sequence of the cytochrome P450 reductase derived from Catharanthous roseus and encoded by the CrCPR gene.

SEQ ID NO: 19 is the amino acid sequence of the aromatic prenyltransferase AtaPT derived from Aspergillus terreus.

SEQ ID NO: 20 is the amino acid sequence of the 7-dimethylallyltryptophan synthase (7-DMATS) derived from Neosartorya fumigatus.

SEQ ID NO: 21 is the amino acid sequence of the phenylpropane-specific prenyltransferase AcPT1 derived from Artemisia capillaris.

SEQ ID NO: 22: is the amino acid sequence of the (R)-(+)-linalool synthase derived from Mentha citrata and encoded by the McLiS gene.

SEQ ID NO: 23: is the amino acid sequence of the (R)-(+)-linalool synthase derived from Mentha citrata and encoded by the McLiS gene and provided with the SKL peroxisomal localization signal.

SEQ ID NO: 24: is the amino acid sequence of the(S)-(−)-limonene synthase derived from Mentha spicata and encoded by the MsLimS gene.

SEQ ID NO: 25: is the amino acid sequence of the(S)-(−)-limonene synthase derived from Mentha spicata and encoded by the MsLimS gene and provided with the SKL peroxisomal localization signal.

SEQ ID NO: 26: is the amino acid sequence of the beta-myrcene synthase derived from Ocimum basilicum and encoded by the ObMyrS gene and provided with the SKL peroxisomal localization signal.

SEQ ID NO: 27: is the amino acid sequence of the limonene-3-hydroxylase derived from Mentha spicata and encoded by the MsLim3H gene.

SEQ ID NO: 28: is the amino acid sequence of the cytochrome P450 reductase derived from Taxus cuspidata and encoded by the tcCPR gene.

SEQ ID NO: 29: is the amino acid sequence of the geranyldiphosphate:olivetolate geranyltransferase derived from Cannabis sativa and encoded by the CsPT4 gene, and provided with the N-terminal peroxisomal localization signal.

### DEFINITIONS AND ABBREVIATIONS

Branch point molecule: A branch point molecule is according to the invention intended to mean a molecule in a biochemical pathway that can be converted into two or more different other molecules or pathways. An example is GPP that can be converted into FPP and thereby be directed into the synthesis of sesqui- and higher terpenes, or it can be converted into a monoterpene by a monoterpene synthase, into cannabinoids by a prenyltransferase enzyme, or into a prenylated aromatic compound by a corresponding prenyltransferase. For branch point molecules there will typical exist a favored or prioritized pathway, which in the natural yeast cells is favored due to e.g. biosynthetic need; and one or more other pathways that are non-prioritized.

DMAPP and IPP: Dimethylallyl pyrophosphate (or dimethylallyl diphosphate; DMAPP) and isopentenyl pyrophosphate (or isopentenyl diphosphate; IPP) are 5-carbon precursors which are used to make isoprenoids

GPP: Geranyl diphosphate (or geranyl pyrophosphate; GPP). GPP is formed by condensation of a DMAPP and an IPP molecule. GPP is a branch point molecule in isoprenoid synthesis and it can, by addition of an IPP molecule, be converted into FPP, and thereby be directed into the biosynthesis of sesqui-, di- or tri-terpenes or sterol synthesis, or it can, by the action of a monoterpene synthase, be directed into the synthesis of monoterpenoids, iridoids, and monoterpene indole alkaloids. Other prenyltransferases can also direct GPP towards the production of cannabinoids, prenylated aromatic compounds, or meroterpenoids in general.

FPP: Farnesyl pyrophosphate (or farnesyl diphosphate; FPP) is formed by condensing GPP with an IPP molecule. FPP is the precursor for the synthesis of sesquiterpenes, diterpenes, triterpenes and sterols.

GGPP: Geranylgeranyl pyrophosphate (or geranylgeranyl diphosphate; GGPP). GGPP is formed by condensing an FPP with an IPP molecule. GGPP is precursor for the synthesis of diterpenes.

Higher terpenes: are in this application intended to mean molecules comprising more than 10 carbon atoms of isoprenoid structure. Examples include sesquiterpenes, diterpenes and triterpenes. Higher terpenes may include moieties not having the isoprenoid structure in addition to the terpene structure.

Monoterpenes: Monoterpenes (or monoterpenoids) are molecules comprising a 10-carbon isoprenoid structure. Monoterpenoids may, in addition to the 10-carbon isoprenoid structure, comprise moieties not having isoprenoid structure. Frequently, the biosynthesis of monoterpenoids involves several additional steps following the initial conversion of GPP to the basic monoterpene skeleton. These additional steps may be oxidations (e.g. catalyzed by a cytochrome P450 enzyme), reductions, isomerizations. acetylations, methylations, etc.

Iridoids: are a group of compounds found in plants and some animals, which are biosynthetically derived from 8-oxogeraniol.

Monoterpene indole alkaloids are a large and diverse group of plant chemical compounds derived from a unit of tryptamine and a 10-carbon or 9-carbon unit of terpenoid origin that is, in turn, derived from 8-oxo-geraniol.

Cannabinoids: are a group of compounds members of which were initially isolated from the plant Cannabis sativa. Many cannabinoids are bio-synthesized by the addition of

GPP to olivetolic acid.

MEP pathway: The methylerythritol 4-phosphate (MEP) pathway forming IPP and DMAPP. The pathway is found e.g. in most bacteria, in algae and is the plastids of higher plants.

MVA pathway: The mevalonate pathway (MVA pathway) is an essential metabolic pathway present in eukaryotes and in some bacteria forming IPP and DMAPP starting from acetyl-CoA.

Alternative MVA pathway: The alternative MVA pathway is found in archaea and provides IPP and DMAPP, starting from acetyl-CoA but utilizing isopentenyl phosphate as intermediate.

Monoterpene synthases. The term includes any enzyme that is able to catalyze the rearrangement of GPP into monoterpenoids. Monoterpene synthases typically synthesize multiple products, but the diversity of products varies among terpene synthases. Some terpene synthases have high product specificity, catalyzing the synthesis of a limited number of products, and other terpene synthases have low product specificity, catalyzing the synthesis of a large variety of different terpenes. Examples of the products of monoterpene synthases include, but are not limited to, the following compounds: tricyclene, alpha-thujene, alpha-pinene, alpha-fenchene, camphene, sabinene, beta-pinene, myrcene, delta-2-carene, alpha-phellandrene, 3-carene, 1,4-cineole, alpha-terpinene, beta-phellandrene, 1,8-cineole, limonene, (Z)-beta-ocimene, (E)-beta-ocimene, gamma-terpinene, terpinolene, linalool, perillene, allo-ocimene, cis-beta-terpineol, cisterpine-1-ol, isoborneol, delta-terpineol, borneol, chrysanthemol, lavandulol, alpha-terpineol, nerol, geraniol. In addition to GPP, certain terpene synthases (or terpene synthase variants developed by protein engineering) have been reported to convert noncanonical prenyl diphosphate substrates, such as the 11-carbon substrate 2-methyl-GPP, to terpenes with non-canonical prenyl scaffolds (Ignea et al. 2018). In the context of this disclosure, enzymes that are able to convert non-canonical prenyl-diphosphates with carbon lengths that differ from 10 into non-canonical terpenoids with 8, 9, 11, or 12 carbons are also included in the definition of monoterpene synthases.

Prenyltransferases: Are enzymes that append a prenyl moiety to isoprenoid or non-isoprenoid skeletons. Many prenyltransferases that append a prenyl moiety to other isoprenoid chains are involved in the synthesis of the prenyl diphosphate precursors, such as GPP (GPP synthases), FPP (FPP synthases), GGPP (GGPP synthases) or geranyl-farnesyl diphosphate synthases (GFPP synthases). These enzymes typically add IPP units to extend DMAPP to larger size prenyl-diphosphates in the trans-configuration. For this reason they are also called trans-polyprenyl synthases or trans-polyprenyl-transferases. Several prenyltransferase enzymes exist that catalyze the cis-condensation and elongation of DMAPP with IPP. These enzymes are termed cis-prenyltransferase, or cis-polyprenyl diphosphate synthase, or cis-polyprenyltransferases, are responsible for the synthesis of neryl diphosphate, cis, cis-farnesyl diphosphate, and nerylneryl diphosphate.

Furthermore, certain isoprenoid prenyltransferases have been reported to condense two DMAPP molecules to lavandulyl diphosphate or chrysanthemyl diphosphate.

Prenyltransferases that append a prenyl moiety to non-isoprenoid scaffolds add DMAPP, GPP, FPP or GGPP to non-isoprenoid compounds, including flavonoids, amino acid residues and peptides, aromatic compounds, and other chemical compounds in general. Such prenyltransferase enzymes are involved in the biosynthesis many different natural products including, but not limited to, cannabinoids, prenylated flavonoids, or other meroterpenoids. In the case of cannabinoid synthesis, this enzyme is a geranyldiphosphate:olivetolate geranyltransferase.

The prenylransferase may be part of separate polypeptides or fused into one polypeptide chain. The prenyltransferase may also be fused to a GPP synthase, a terpene synthase, or another non-terpene synthesizing protein. The prenyltransferase may also be fused to an enzyme that naturally localizes to the peroxisome matrix or its membrane in yeasts or in another organism, or that it is fused to a polypeptide chain that is itself fused to a peroxisomal targeting signal.

An aromatic prenyltransferase is selected among any enzyme with prenyltransferase activity, identified from any organism or engineered, that is able to transfer an isoprenoid moiety to another isoprenoid or non-isoprenoid compound. The prenylransferase may be part of separate polypeptides or fused into one polypeptide chain. The prenyltransferase may also be fused to a GPP synthase, a terpene synthase, or another non-terpene synthesizing protein. The prenyltransferase may also be fused to an enzyme that naturally localizes to the peroxisome matrix or its menbrane in yeasts or in another organism, or that it is fused to a polypeptide chain that is itself fused to a peroxisomal targeting signal.

### DETAILED DESCRIPTION OF THE INVENTION

The invention is based on the observation that in biochemical pathways branch points exist where branch point molecules can be diverted into different biochemical pathways and that the prioritized pathway leads to compounds that are mandatory for growth of the particular organism, whereas other pathways are not prioritized (designated nonprioritized pathway). It follows that special measures are required to make the cell prioritize a non-prioritized pathway.

According to the invention, the production of a compound of a non-prioritized pathway is increased by peroxisomal localization of an enzyme catalyzing the formation of the branch point compound and an enzyme catalyzing the first step of the non-prioritized pathway.

The enzyme catalyzing the formation of a branch point compound and the enzyme catalyzing the first step of the non-prioritized pathway may be present as separate molecules, they may be present as a single molecule comprising a domain catalyzing the formation of a branch point compound and another domain that catalyzes the first step of the non-prioritized pathway or they may even be present in form of a multidomain molecule that in addition to one or two of the two enzymatic activities comprise one or more additional domains with different function.

GPP is such a branch point molecule in terpene synthesis and it can be converted into monoterpenoid or compounds comprising a 10-carbon monoterpenoid structure attached to a non-prenyl moiety; into sesqui-, di- or tri-terpenoids or compounds comprising a 15-carbon sesqui-, 20-carbon di- or a 30-carbon tri-terpenoid structure; or into sterols.

In yeasts, sterols are essential for the growth and survival of the cells, therefore the conversion of GPP into FPP, and eventually into sterols, is prioritized.

Thus, in a first aspect, the invention relates to a yeast cell wherein an enzyme catalyzing the formation of GPP and an enzyme catalyzing the first step in a pathway starting from GPP and forming a monoterpenoid, cannabinoid, iridoid, monoterpene indole alkaloid, or a prenylated aromatic compound are localized to the peroxisomes.

Enzymes catalyzing the formation of GPP, also called GPP synthases, are known for the skilled person. The invention is not limited to any particular GPP synthase, so, in principle, any GPP synthase may be peroxisomally localized and used according to the invention. The GPP synthase may be a homologous GPP synthase, i.e. an enzyme originating from the same species as the host cell, it may be a heterologous enzyme, i.e. an enzyme that originate from a different species than the host cell, or it may even be a synthetic enzyme, i.e. an enzyme that does not occur in nature but is artificially created using techniques known in the art of genetic engineering. The GPP synthase can be a single subunit, or multi-subunit enzyme that is composed from identical or non-identical subunits (several such examples exist in nature and are known to the skilled person, i.e. the combination of the large with the small subunit of snapdragon GGPP synthase (Orlova I. et al 2009)). The subunit(s) of the GPP synthase may be part of separate polypeptides or fused into one polypeptide chain. The subunit(s) may also be fused to a terpene synthase, a prenyltransferase, or another non-terpene synthesizing protein. In particular, it may be fused to an enzyme that naturally localizes to the peroxisome in yeasts or in another organism, or that it is fused to a polypeptide chain that is, in turn, fused to a peroxisomal targeting signal.

An example of a preferred GPP synthase according to the invention is an engineered GPP synthase Erg20p^{N127W} (SEQ ID NO: 1) which is the native S. cerevisiae GPP synthase containing the substitution N127W. The N127W substitution blocks the catalytic site of the enzyme to prevent further conversion of GPP into FPP by addition of an IPP molecule.

Other preferred GPP synthases includes polypeptides having, alone or in combination with other polypeptides, GPP synthase activity, said polypeptides originating from an organism that belongs to any of the kingdoms of life, i.e. Bacteria, Archaea, Protozoa, Chromista, Plantae, Fungi, or Animalia. Other preferred synthases include enzymes that have been engineered to have, alone or in combination with other polypeptides, GPP synthase activity, using protein engineering.

Enzyme catalyzing the first step in a pathway starting from GPP and forming a monoterpenoid, iridoid, cannabinoid, monoterpene indole alkaloid, a prenylated aromatic compound, or other meroterpenoids; are also known for the person skilled in the art. Non-limiting examples include monoterpene synthases, such as (+)-limonene synthase, (−)-limonene synthase, α-pinene synthase, 1,8-cineole synthase, sabinene synthase, camphene synthase, linalool synthase, myrcene synthase, or geraniol synthase, and prenyltransferases, such an geranyldiphosphate:olivetolate geranyltransferase, or a broad specificity aromatic prenyltransferase.

Examples of preferred monoterpene synthase according to the invention includes the (+)-limonene synthase derived from Citrus limon and having the amino acid sequence of SEQ ID NO: 3; the camphene synthase derived from Solanum elaeagnifolium and having the amino acid sequence of SEQ ID NO: 5; the (−)-limonene synthase derived from Mentha spicata and having the amino acid sequence of SEQ ID NO: 25; the (+)-linalool synthase derived from Mentha citrata and having the amino acid sequence of SEQ ID NO: 23; the myrcene synthase derived from Ocimum basilicum and having the amino acid sequence of SEQ ID NO: 26; the α-pinene synthase derived from Pinus taeda and having the amino acid sequence of SEQ ID NO: 7; the sabinene synthase derived from Salvia pomifera and having the amino acid sequence of SEQ ID NO: 9; and the geraniol synthase form Ocimum basilicum and having the amino acid sequence of SEQ ID NO: 11

Other preferred monoterpene synthases includes polypeptides having activity as betapinene synthase, (−)-limonene synthase, linalool synthase, myrcene synthase, bornyl diphosphate synthase, alpha-terpineol synthase, isoborneol synthase, tricyclene synthase, alpha-thujene synthase, alpha-fenchene synthase, delta-2-carene synthase, alpha-phellandrene synthase, 3-carene synthase, 1,4-cineole synthase, alpha-terpinene synthase, beta-phellandrene synthase, 1,8-cineole synthase, (Z)-beta-ocimene synthase, (E)-beta-ocimene synthase, gamma-terpinene synthase, terpinolene synthase, allo-ocimene synthase, cis-beta-terpineol synthase, cis-terpine-1-ol synthase, delta-terpineol synthase, borneol synthase, alpha-terpineol synthase, nerol synthase, 2-methylisoborneol synthase, 2-methylenebornene synthase, 2-methyl-2-bornene synthase, or beta-phellandrene synthase.

Prenyltransferases able to attach the geranyl moiety to a non-isoprenoid scaffold include the geranyldiphosphate: olivetolic geranyltransferase CsPT4 derived from Cannabis sativa and having the amino acid sequence of SEQ ID NO: 13, or the aromatic prenyltransferase AtaPT from Aspergillus terreus and having the amino acid sequence of SEQ ID NO: 19.

Other preferred prenyltransferases able to add the prenyl group to isoprenoid scaffolds include neryl diphosphate synthase, chrysanthemyl diphosphate synthase, or lavandulyl diphosphate synthase, while preferred prenyltransferases able to append the prenyl group to non-isoprenoid scaffolds include the 7-dimethylallyltryptophan synthase from Aspergillus fumigatus (7-DMATS) (SEQ ID NO: 20) and the phenylpropane-specific prenyltransferase AcPT1 from Artemisia capillaris (SEQ ID NO: 21).

In one preferred embodiment, a Saccharomyces cerevisiae cell is provided wherein a GPP synthase and a limonene synthase are localized to the peroxisomes. The inventors have found that this alone is sufficient to induce a 32-fold improvement in the monoterpene (limonene) production compared with the production obtained when these two enzymes are expressed in the cytosol, or only one of the two enzymes is present in the peroxisome and the other in the cytosol. Further, additional peroxisomal compartmentalization of the complete MVA pathway, comprised of EfmvaS, EfmvaE, Erg12p, Erg9p and Idi1p improved the monoterpene production by 14-fold, 17-fold, 17-fold, 20.5-fold, 22-fold, and 125 fold, respectively for camphene, pinene, (−)-limonene, (+)-linalool, sabinene and (+)-limonene, compared with an identical yeast cell where the enzymes are localized in the cytosol.

In another preferred embodiment, a S. cerevisiae cell is provided wherein a GPP synthase and a geraniol synthase are localized to the peroxisomes. The yeast cell produces improved level of geraniol, a precursor for iridoids and monoterpene indole alkaloids, compared with same cell wherein the enzymes are localized in the cytosol. In a further preferred embodiment, a S. cerevisiae cell is provided, wherein a GPP synthase and an olivetolic acid prenyltransferase are localized to the peroxisomes. The yeast cell is efficient in producing cannabigerolic acid, the precursor of several cannabinoid compounds.

In a further embodiment a S. cerevisiae cell is provided, wherein a GPP synthase and the aromatic prenyltransferase AtaPT, from Aspergillus terreus are localized to the peroxisomes. When provided with umbeliferone, quercetin, isoquercetin, resveratrol, or naringenin, the yeast cell afforded efficient synthesis of osthrutin, geranylated quercetin, geranylated isoquercetin, geranylresveratrol, and geranyl-naringenin respectively. In a further embodiment, a S. cerevisiae cell is provided, wherein an isopentenyl diphosphate isomerase (IDI), which is a DMAPP synthesizing enzyme, and a terpene synthase catalyzing the synthesis of isoprene (isoprene synthase; ISPS) are localized to the peroxisomes. The yeast cell produced afforded efficient synthesis of isoprene. In a further embodiment, a S. cerevisiae cell is provided, wherein an isopentenyl diphosphate isomerase (IDI) and the lavandulyl diphosphate synthase from Lavandula x intermedia are localized to the peroxisomes. The yeast cell afforded efficient synthesis of lavandulol.

In a further embodiment, a S. cerevisiae cell is provided, wherein an isopentenyl diphosphate isomerase (IDI) and the chrysanthemyl diphosphate synthase from Tanacetum cinerariifolium are localized to the peroxisomes. The yeast cell afforded efficient synthesis of chrysanthemol.

In a further embodiment, a S. cerevisiae cell is provided, wherein an isopentenyl diphosphate isomerase (IDI) and the 7-dimethylallyltryptophan synthase from Aspergillus fumigatus (7-DMATS) are localized to the peroxisomes. The yeast cell afforded efficient synthesis of prenyl-tryptophan.

In a further embodiment, a S. cerevisiae cell is provided, wherein an isopentenyl diphosphate isomerase (IDI) and the phenylpropane-specific prenyltransferase AcPT1 from Artemisia capillaris are localized to the peroxisomes. When provided with p-coumaric acid, the yeast cell afforded efficient synthesis of drupanin and artepillin C.

In a further embodiment, a S. cerevisiae cell is provided, wherein an isopentenyl diphosphate isomerase (IDI) and the O-prenyltrasferase AcaPT from Antrodia camphorata are localized to the peroxisomes. When provided with apigenin, kaempherol, daidzein, naringenin, genistein, isoliquiritigenin, equol, umbelliferone, curcumin, resveratrol, or diethylstilbestrol, the yeast cell afforded efficiently synthesis of 4′-dimethylallyl-apigenin, 4′-dimethylallyl-naringenin, 4′-dimethylallyl-kaempferol, 4′-dimethylallyl-daidzein, 7-dimethylallyl-daidzein, 7,4′-di-(dimethylallyl)-daidzein, 4′-dimethylallyl-genistein, 7-dimethylallyl-genistein, 7,4′-di-(dimethylallyl)-genistein, 4-dimethylallyl-isoliquiritigenin, 4′-dimethylallyl-equol, 7-dimethylallyl-equol, 6-dimethylallyl-equol, 4′-dimethylallyl-daidzin, 7-dimethylallyl-umbelliferone, 8-dimethylallyl-curcumin, 8′-dimethylallyl-demethoxycurcumin, 8-dimethylallyl-demethoxycurcumin, 7-dimethylallyl-Ltryptophan, 4′-dimethylallyl-resveratrol, or 5-dimethylallyl-diethylstilbestrol.

### Peroxisomal Localization

According to the invention, the expression peroxisomal localization or grammatically equivalent terms in connection with biosynthetic enzymes for the terpene pathways, is intended to mean that the enzymes in question are translocated to the peroxisomes or the peroxisome membrane after synthesis and that the enzymes thereafter exerts their catalytic functions in the peroxisomes.

Peroxisomal localization can be effectuated by providing the gene encoding the enzyme to be peroxisomally localized with a peroxisomal localization signal. Peroxisomal localization and peroxisomal localization signals are known in the art e.g. in WO9424289A1 and KR101308971B1 (incorporated herein by reference); and such signals and methods known in the art are also useable according to the present invention.

A preferred peroxisomal localization signal is SKL (SerLysLeu) added to the C-terminus of the polypeptide to be peroxisomally localized or any C-terminal tripeptide with the canonical sequence (S/A/C)-(K/R/H)-(L/M).

For yeast, another preferred localization signal consists of the conserved peptide (R/K)(L/V/I)-X₅-(H/Q)-(L/A/F) added to the N-terminus of the polypeptide to be peroxisomally localized.

An additional method to effectuate peroxisomal localization of a protein is to fuse said protein with another protein that is naturally found in the peroxisomes of yeasts or other organisms. A further method to achieve peroxisomal localization of a protein is to construct a protein fusion between said protein and another protein (or protein domain) that does not normally reside in the peroxisome but is engineered to localize to the peroxisome by the addition of a localization signal as described above.

The peroxisomally localized enzymes may be homogeneous, meaning that the peroxisomally localized enzyme is identically to enzyme naturally found in the cytoplasm of the host cell or it may be heterologous, meaning that it is different from the enzyme naturally found in the cytoplasm of the host cell.

According to the invention, peroxisomal localization of an enzyme means that a gene encoding the enzyme in question, provided with an encoded peroxisomal localization signal is introduced into the host cell. If the enzyme in question is an enzyme that is naturally found in the host cell, it is believed that the peroxisomally localized enzyme provides for the improved synthesis of monoterpenoids, cannabinoids, iridoids monoterpene indole alkaloids and other prenylated compounds according to the invention, whereas the naturally enzyme localized in the cytoplasm of the host cell provides for the normal biosynthesis of biomolecules necessary for survival and growth of the host cell. For example, if the enzyme in question is a GPP synthase, the peroxisomally localized GPP synthase will provide the improved synthesis of monoterpenoids, cannabinoids, iridoids, monoterpene indole alkaloids, and other prenylated compounds according to the invention and the natural GPP-synthesizing enzyme localized in the cytoplasm will secure that GPP is provided for the biosynthesis of required molecules e.g. sterols; necessary to secure survival and normal growth of the host cell.

If the selected host cell is a polyploid cell, e.g. a diploid or tetraploid cell; it may even be possible to provide for peroxisomal localization by gene editing techniques resulting that one or more allele of the gene encoding the enzyme in question is provided with a peroxisomal localization signal and leaving at least one allele unaltered. This will secure that the edited allele(s) provide for the peroxisomal localized enzyme, whereas the non-edited allele(s) provides for the natural enzyme localized in the cytoplasm.

### Host Cells

The host cell is according to the invention a yeast cell, i.e. a eukaryotic single cellular organism; reviewed e.g. in: The yeasts. 5^{th} edition. A taxonomic study. Editors: Kurtzman, Fell, Boekhout. Elsevier, 2011.

Preferred host cells include cells belonging to the genera: Saccharomyces, Pichia, Candida, Yarrowia, Ogataea. More preferred, the host cells are selected among the species; Saccharomyces cerevisiae, Pichia pastoris, Yarrowia lipolytica, Ogataea polymorpha, Candida albicans, Candida boidinii.

The invention is not limited to any particular methods for providing the peroxisomal localization for the enzymes according to the invention. Any method known in the art for providing suitable genes, optimizing codon usage, providing suitable regulatory elements such as promoters, terminators, adenylation sites, introns, exons, enhancer elements, ribosome binding sites, Kozak sequences, transforming yeasts, etc. may be used according to the invention.

### Production

The invention is also related to the production of monoterpenoids, cannabinoids, iridoids, monoterpene indole alkaloids and other prenylated compounds using a yeast cell of the invention.

According to the invention monoterpenoids, cannabinoids, iridoids, monoterpene indole alkaloids, and prenylated compounds may be produced using a method comprising the steps of:
a. Providing a yeast cell according to the invention;
b. Growing the yeast cell in a substrate supporting growth of the yeast cell; and
c. When required, providing a co-substrate to be prenylated.
d. Recovering the compound from the fermentation broth, or converting the compound to more complex products within the yeast cells by the action of additional native or heterologously expressed enzymes.

The compound provided by this method may even be further converted to more complex products within the yeast cells by the action of additional native or heterologously expressed enzymes.

Growing the yeast can in principle be done in any known method for growing yeast, but in order to facilitate the recovery it is preferred to grow the yeast cells in liquid medium in a container such as in shake-flasks or fermenters.

It is particular preferred to grow the yeast cells in a fermenter and the fermentation process may be performed as a batch fermentation, fed-batch fermentation, or a continuous fermentation, as known in the art.

The substrate supporting growth of the yeast cell may be any suitable medium comprising a carbon source, nitrogen source, minerals and nutritionals required by the particular yeast cell.

The substrate may be a complex substrate comprising ingredients that are not fully defined, or it may be a defined medium comprising only defined ingredients.

As examples of ingredients for a complex medium can be mentioned molasses, dextrins, hydrolysates of starch and/or proteins.

As examples of ingredients for a defined medium can be mentioned: glucose, sucrose, ammonia, salts, minerals and vitamins.

The fermentation process generates a fermentation broth comprising cells, water, product, remaining nutritionals and minerals and waste products generated by the cells. The recovery of the monoterpenoids, cannabinoids, iridoids, monoterpene indole alkaloids, and other prenylated compounds from the fermentation broth is done using methods known in the art for recovering such compounds.

### Materials and Methods

### Genes Used:

TABLE 1
List of genes used in the study. Accession numbers are for
Uniprot.org. Where unavailable a reference is cited.

| | | | Accession number/ |
|---|---|---|---|
| Gene | Origin | Function | reference |
| EfmvaS | Enterococcus faecalis | HMG-CoA synthase | Q9FD71 |
| EfmvaE | Enterococcus faecalis | Acetyltransferase + | Q9FD70 |
| | | HMG-CoA reductase | |
| ERG8 | S. cerevisiae | Phosphomevalonate kinase | P24521 |
| ERG12 | S. cerevisiae | Mevalonate kinase | P07277 |
| ERG19 | S. cerevisiae | Diphosphomevalonate | P32377 |
| | | decarboxylase | |
| IDI1 | S. cerevisiae | Isopentenyl-diphosphate | P15496 |
| | | Delta-isomerase | |
| ERG20 | S. cerevisiae | Farnesyl pyrophosphate | P08524 |
| | | synthase | |
| ERG20^{N127W} | S. cerevisiae | Geranyl pyrophosphate | (Ignea et al 2014) |
| | | synthase | |
| C/LimS | Citrus limon | (+)-limonene synthase | Q8L5K3 |
| PiPinS | Pinus taeda | α-pinene synthase | Q84KL3 |
| SpSabS | Salvia pomifera | Sabinene synthase | A6XH06 |
| SeCamS | Solarium elaeagnifolium | Camphene synthase | (Philips et al 2003) |
| CrG8OH | Catharanthous roseus | geraniol 8-hydroxylase | Q8VWZ7.1 |
| MsLim3H | Mentha spicata | Limonene-3-hydroxylase | Q6IV13.1 |
| CrCPR/POR | Catharanthous roseus | cytochrome P450 reductase | Q05001.1 |
| tcCPR/POR | Taxus cuspidate | cytochrome P450 reductase | AAT76449.1 |
| McLiS | Mentha citrata | (R)-(+)-linalool synthase | AAL99381.1 |
| MsLimS | Mentha spicata | (S)-(−)-limonene synthase | AAC37366.1 |
| ObMyrs | Ocimum basilicum | Myrcene synthase | Q5SBP1.1 |

### Yeast Strains

The yeast strains used in this application were based on the EGY48 Saccharomyces cerevisiae strain disclosed in (Ignea et al (2011), Thomas B. J. and R. Rothstein (1989) and (Ellerstrom M et al (1992)), and modified according to Table 2.

TABLE 2
Strains

| Strain | Genotype |
|---|---|
| EGY48 | MATα, ura3, his3, trp1, 6xLexA operators::LEU2 - Derivative of the U457 |
| | strain (W303-1A background) |
| PERLim06 | EGY48 - P_{Gal1}-Efmva-SKL-tPGI1, P_{Gal10}-EfmvaE-SKL-tCYC1, P_{SED1}-ERG12-SKL-tFBA1, |
| | P_{Tdh3}-ERG8-SKL-tPRM8, P_{Fba1}-ERG19-SKL-tSPG5, P_{Cww12}-IDI-SKL-tENO2, |
| | P_{Tef1}-ERG20^{N127W}-SKL-tTDH2, P_{Pgk1}-LimS-SKL-tADH1. |
| PERGer01 | EGY48 - P_{Gal1}-Efmva-SKL-tPGI1, P_{Gal10}-EfmvaE-SKL-tCYC1, P_{SED1}-ERG12-SKL-tFBA1, |
| | P_{Tdh3}-ERG8-SKL-tPRM8, P_{Fba1}-ERG19-SKL-tSPG5, P_{Cww12}-IDI-SKL-tENO2, |
| | P_{Tef1}-ERG20^{N127W}-SKL-tTDH2, P_{Pgk1}-tOBGES-SKL-tADH1. |
| CYTMva01 | EGY48 - P_{Gal1}-Efmva-tPGI1, P_{Gal10}-EfmvaE -tCYC1, P_{SED1}-ERG12 -tFBA1, |
| | P_{Tdh3}-ERG8 -tPRM8, P_{Fba1}-ERG19-tSPG5, P_{Cww12}-IDI-tENO2, |
| PERMva01 | EGY48 - P_{Gal1}-Efmva-SKL-tPGI1, P_{Gal10}-EfmvaE-SKL-tCYC1, P_{SED1}-ERG12-SKL-tFBA1, |
| | P_{Tdh3}-ERG8-SKL-tPRM8, P_{Fba1}-ERG19-SKL-tSPG5, P_{Cww12}-IDI-SKL-tENO2, |

### Constructions of Plasmids:

Plasmids were generated using standard methods used within genetic engineering and known in the art. Detailed protocols for methods for plasmid constructions can be found in general handbooks containing methods for molecular cloning.

Plasmids designed to provide for peroxisomal localization of enzymes, named pPER includes the peroxisomal localization signal (-SKL) fused C-terminally to the amino acid sequence of the enzymes or an N-terminal peroxisomal localization signal, whereas plasmids designed to provide cytoplasmatic localization of enzymes (pCYT) did not contain this signal.

Genes were amplified by PCR and placed under the control of the dual inducible promoter P_{GAL1} and P_{GAL10}. Coding genes sequences were then ligated using USER cloning (Nour-Eldin et al (2010)) into the backbone of the pESC-URA, pESC-LEU, pESCTRP, and pESC-HIS, vectors (Agilent Technologies) to construct the plasmids listed in Table 3.

TABLE 3
List of plasmids used in the study

| Plasmid | Description |
|---|---|
| pCYT01 | 2μ; TRP1; AmpR; P_{Gal1}-EfmvaS-tAHD1; P_{Gal10}-EfmvaE-tCYC1 |
| pCYT02 | 2μ; HIS3; AmpR; P_{Gal1}-ERG8-tAHD1; P_{Gal10}-ERG12-tCYC1 |
| pCYT03 | 2μ; LEU2; AmpR; P_{Gal1}-IDI-tAHD1; P_{Gal10}-ERG19-tCYC1 |
| pCYT04 | 2μ; URA3; AmpR; P_{Gal1}-C/LimS-tAHD1; P_{Gal10}-ERG20^{N127W}-tCYC1 |
| pCYT05 | 2μ; URA3; AmpR P_{Gal1}-PtPinS-tAHD1; P_{Gal10}-ERG20^{N127W}-tCYC1 |
| pCYT06 | 2μ; URA3; AmpR P_{Gal1}-SpSabS-tAHD1; P_{Gal10}-ERG20^{N127W}-tCYC1 |
| pCYT07 | 2μ; URA3; AmpR; P_{Gal1}-SeCamS -tAHD1; P_{Gal10}-ERG20^{N127W}-tCYC1 |
| pCYT08 | 2μ; HIS3; AmpR; P_{Gal1}-C/LimS-tAHD1 |
| pCYT09 | 2μ; TRP1; AmpR; P_{Gal1}-CYP71D95-tAHD1; P_{Gal10}-fcCPR-tCYC1 |
| pCYT10 | 2μ; TRP1; AmpR; P_{Gal1}-CYP71D95-tAHD1; P_{Gal10}-crCPR-tCYC1 |
| pCYT11 | 2μ; TRP1; AmpR; P_{Gal1}-CYP71D95-tAHD1; P_{Gal10}-hpCPR-tCYC1 |
| pCYT12 | 2μ; URA3; AmpR; P_{Gal1}-McLiS -tAHD1; P_{Gal10}-ERG20^{N127W}-tCYC1 |
| pCYT13 | 2μ; URA3; AmpR; P_{Gal1}-MsLimS -tAHD1; P_{Gal10}-ERG20^{N127W}-tCYC1 |
| pCYT14 | 2μ; URA3; AmpR; P_{Gal1}-CsPT4-tAHD1; P_{Gal10}-ERG20^{N127W}-tCYC1 |
| pPER01 | 2μ; TRP1; AmpR; P_{Gal1}-EfmvaS-SKL-tAHD1; p_{Gal10}-EfmvaE-SKL-tCYC1 |
| pPER02 | 2μ; HIS3; AmpR; P_{Gal1}-ERG8-SKL-tAHD1; P_{Gal10}-ERG12-SKL-tCYC1 |
| pPER03 | 2μ; LEU2; AmpR; P_{Gal1}-IDI-SKL-tAHD1; P_{Gal10}ERG19-SKL-tCYC1 |
| pPER04 | 2μ; URA3; AmpR; P_{Gal1}-C/LimS-SKL-tAHD1; P_{Gal10}-ERG20^{N127W}-SKL-tCYC1 |
| pPER05 | 2μ; URA3; AmpR; P_{Gal1}-PtPinS-SKL-tAHD1; P_{Gal10}-ERG20^{N127W}-SKL-tCYC1 |
| pPER06 | 2μ; URA3; AmpR; P_{Gal1}-SpSabS-SKL-tAHD1; P_{Gal10}-ERG20^{N127W}-SKL-tCYC1 |
| pPER07 | 2μ; URA3; AmpR; P_{Gal1}-SeCamS-SKL-tAHD1; P_{Gal10}-ERG20^{N127W}-SKL-tCYC1 |
| pPER08 | 2μ; URA3; AmpR; P_{Gal1}-C/LimS-SKL-tAHD1 |
| pPER09 | 2μ; HIS3; AmpR; P_{Gal1}-ERG8-SKL-tAHD1 |
| pPER10 | 2μ; HIS3; AmpR; P_{Gal1}-ERG12-SKL-tAHD1 |
| pPER11 | 2μ; LEU2; AmpR; P_{Gal1}-ERG20^{N127W}-SKL-tAHD1 |
| pPER12 | 2μ; LEU2; AmpR; P_{Gal1}-C/LimS-SKL-tAHD1 |
| pPER13 | 2μ; URA3; AmpR; P_{Gal1}-ObGES-SKL-tAHD1; P_{Gal10}-ERG20^{N127W}-SKL-tCYC1 |
| pPER14 | 2μ; URA3; AmpR; P_{Gal1}-CsPT4-SKL-tAHD1; P_{Gal10}-ERG20^{N127W}-SKL-tCYC1 |
| pPER15 | 2μ; URA3; AmpR; P_{Gal1}-ERG20^{N127W}-5GS-CILimS-SKL-tAHD1 |
| pPER16 | 2μ; URA3; AmpR; P_{Gal1}- C/LimS -5GS- ERG20^{N127W} -SKL-tAHD1 |
| pPER17 | 2μ; URA3; AmpR; P_{Gal1}-crG8OH-tAHD1; P_{Gal10}-crCPR-tCYC1 |
| pPER18 | 2μ; URA3; AmpR; P_{Gal1}-McLiS-SKL -tAHD1; P_{Gal10}-ERG20^{N127W}-SKL-tCYC1 |
| pPER19 | 2μ; URA3; AmpR; P_{Gal1}-MsLimS-SKL -tAHD1; P_{Gal10}-ERG20^{N127W}-SKL-tCYC1 |
| pPER20 | 2μ; URA3; AmpR; P_{Gal1}-ObMyrS-SKL -tAHD1; P_{Gal10}-ERG20^{N127W}-SKL-tCYC1 |
| pPER21 | 2μ; URA3; AmpR; P_{Gal1}-MsLim3H-tAHD1; P_{Gal10}-tcCPR-tCYC1 |
| pPER22 | 2μ; URA3; AmpR; P_{Gal1}-PTS2-CsPT4-tAHD1; P_{Gal10}-ERG20^{N127W}-SKL-tCYC1 |

Designation of Strains Harboring Plasmids Used in this Application

Plasmids (Table 3) were then used to transform yeast cells (Table 2) using the lithium acetate/PEG method. Transformants were selected by their respective auxotrophy on the corresponding minimal media.

TABLE 4
Strains harboring episomal vectors used in the study

| Parent | Strain | Plasmids |
|---|---|---|
| EGY48 | CYTLim01 | pCYT08 |
| EGY48 | CYTLim02 | pCYT01, pCYT02, pCYT03, pCYT04 |
| EGY48 | PERLim01 | pPER08 |
| EGY48 | PERLim02 | pCYT01, pCYT02, pCYT03, pPER04 |
| EGY48 | PERLim03 | pCYT01, pCYT02, pPER03, pPER04 |
| EGY48 | PERLim04 | pCYT01, pPER02, pPER03, pPER04 |
| EGY48 | PERLim05 | pPER01, pPER02, pPER03, pPER04 |
| PERLim06 | PERLim07 | pPER01, pPER02, pPER03, pPER04 |
| PERLim06 | PERLim08 | pPER11 |
| PERLim06 | PERLim09 | pPER04 |
| PERLim06 | PERLim10 | pPER15 |
| PERLim06 | PERLim11 | pPER16 |
| EGY48 | CYTCam01 | pCYT07 |
| EGY48 | CYTPin01 | pCYT05 |
| EGY48 | CYTSab01 | pCYT06 |
| EGY48 | CYTCam02 | pCYT01, pCYT02, pCYT03, pCYT07 |
| EGY48 | CYTPin02 | pCYT01, pCYT02, pCYT03, pCYT05 |
| EGY48 | CYTSab02 | pCYT01, pCYT02, pCYT03, pCYT06 |
| EGY48 | CYTLin01 | pCYT01, pCYT02, pCYT03, pCYT12 |
| EGY48 | CYTLim04 | pCYT01, pCYT02, pCYT03, pCYT13 |
| EGY48 | CYTLim06 | pCYT01, pCYT02, pPER21, pCYT13 |
| CYTMva01 | CYTCan01 | pCYT01, pCYT02, pCYT03, pCYT14 |
| EGY48 | PERCam01 | pPER07 |
| EGY48 | PERPin01 | pPER05 |
| EGY48 | PERSab01 | pPER06 |
| EGY48 | PERCam02 | pPER01, pPER02, pPER03, pPER07 |
| EGY48 | PERPin02 | pPER01, pPER02, pPER03, pPER05 |
| EGY48 | PERSab02 | pPER01, pPER02, pPER03, pPER06 |
| EGY48 | PERLin01 | pPER01, pPER02, pPER03, pPER18 |
| EGY48 | PERLim27 | pPER01, pPER02, pPER03, pPER19 |
| EGY48 | PERLim29 | pPER01, pPER02, pPER19, pESC-Leu |
| EGY48 | PERLim30 | pPER01, pPER02, pPER19, pPER21 |
| EGY48 | PERMyr01 | pPER01, pPER02, pPER03, pPER20 |
| PERGer01 | PERGer02 | pPER01, pPER02, pPER03, pPER13 |
| PERGer01 | PERGer03 | pPER01, pPER02, pPER13, pESC-Leu |
| PERGer01 | PERGer04 | pPER01, pPER02, pPER13, pPER17 |
| PERMva01 | PERCan01 | pPER01, pPER02, pPER03, pPER14 |
| PERMva01 | PERCan02 | pPER01, pPER02, pPER03, pPER22 |

### Culture Conditions

The yeast cells were first cultured on selective minimal media with glucose at 30° C. overnight. Complete minimal media consisted of 0.13% w/v dropout powder, 0.67% w/v yeast nitrogen base without amino acids with ammonium sulphate (YNB+AS), 2% w/v glucose. Dropout powder was purchased to lack leucine, histidine, uracil and tryptophan. When required, these four nutrients were added at 0.01-0.02% w/v. Cells were then harvested by centrifugation to remove medium and resuspended in selective minimal production media with an initial OD_{600 nm} around 0.5. This media was used to induce galactose promoters, with additional raffinose as an alternative carbon source. Media composition: 0.13% w/v dropout powder, 0.64% w/v YNB+AS, 2% galactose, 1% w/v raffinose. When appropriate, the same four nutrients as above were added at 0.01-0.02% w/v. Isopropylmyristate (IPM) was added as an overlay corresponding to 10% of the culture volume. The cultures were grown at 30° C., 150 rpm, for the indicated time, the cells were then harvested by centrifugation, and the IPM phase recovered and analyzed using GC-FID and/or GC-MS.

### EXAMPLES

### Example 1: Peroxisomal Co-Localization of a GPP Synthase and a Terpene Synthase Improves Terpene Production

### Yeast Strain Construction

The Saccharomyces cerevisiae strains used were derived from the strain EGY48 (Mat α, ura3, trp1, his3, 6xLexA operators::LEU2). An engineered GPP synthase, ERG20^{N127W} from S. cerevisiae, and a monoterpene synthase (MTs) characterized as a limonene synthase, C/LimS from Citrus limon, were expressed under the control of the P_{GAL1}-P_{GAL10} promoter.

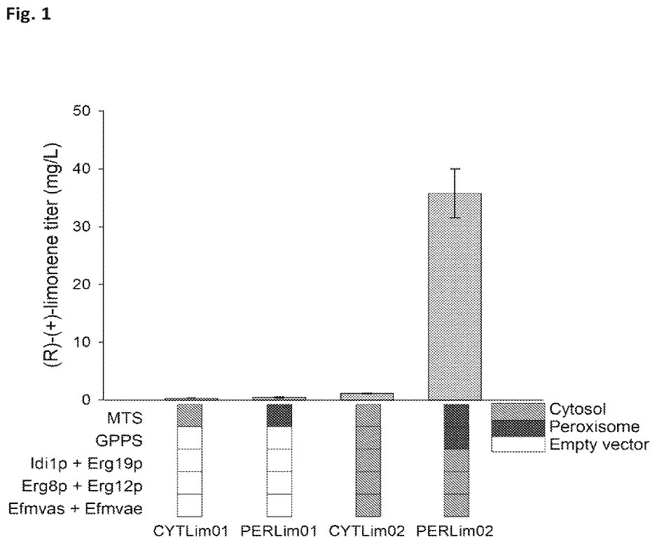

First, the C/LimS was expressed in the cytosol of the strain EGY48 (strain CYTLim01) As shown in FIG. 1, this strain produced only 0.31 mg/L of limonene. A very similar result was observed when targeting C/LimS to the peroxisome by addition of the C-terminal PTS1 SKL (strain PERLim01). Indeed, although GPP could be translocated from the cytosol into the peroxisome, the naturally very low cytosolic GPP pool is most likely to result in a very limited transport of this molecule into other compartments.

Overexpressing the entire MVA pathway in the cytosol using the Enterococcus faecalis EfmvaE and EfmvaS genes (equivalent to Erg10p, Erg13p and HmgRp in yeast), Erg8p, Erg12p, Erg9p, Idi1p and Erg20p^{N127W} (as a GPP synthase) resulted in a 3.6-fold increase in limonene production to 1.12 mg/L (Strain CYTLim02).

However, by only targeting the GPP synthase ERG20p^{N127W} and C/LimS to the peroxisome (strain PERLim02) the limonene production was improved drastically by 32 fold, compared to CYTLim02, to reach 35 mg/L. This indicates that the precursors IPP and/or DMAPP can be transported into the peroxisome and can be converted to GPP in this organelle.

The significant increase in limonene production also shows that the peroxisome is effectively able to act as a barrier and protect this newly formed GPP from the cytosolic ERG20p, thus allowing its uptake by C/LimS.

The results are also shown in FIG. 1.

### Example 2: Localization of the Mevalonate Pathway into Peroxisomes Enhances Production of Terpenoids

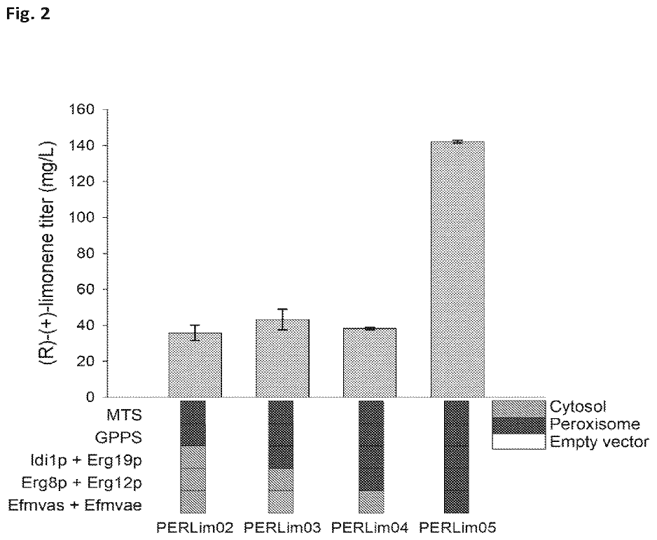

To assess the possibility of harvesting peroxisomal acetyl-CoA for GPP production in this organelle, the eight MVA pathway enzymes were targeted to the peroxisome by addition of a C-terminal peroxisomal targeting signal of type 1 (PTS1) composed of the tripeptide SKL (see sequences part). Although the presence of the GPP synthase Erg20p^{N127W} and C/LimS was sufficient to observe a notable jump in limonene production, stepwise localization of additional enzymes of the MVA pathway to the peroxisome improved limonene production only slightly when the pathway was not complete in this organelle. However, when all eight enzymes were peroxisomally targeted, an additional 4-fold increase in limonene production to 141 mg/L was observed (FIG. 2).

Moving the entire pathway from acetyl-CoA to limonene from the cytosol (strain CYTLim02) to the peroxisome (strain PERLim05) gives an overall 125-fold improvement in the production.

### Example 3: Expanding the Invention to Other Monoterpenoids. Construction of a Yeast Strain for Improved Camphene, Pinene, (S)-(−)-Limonene, (R)-(+)-Limonene, (R)-(+)-Linalool and Sabinene Production Using the Peroxisome

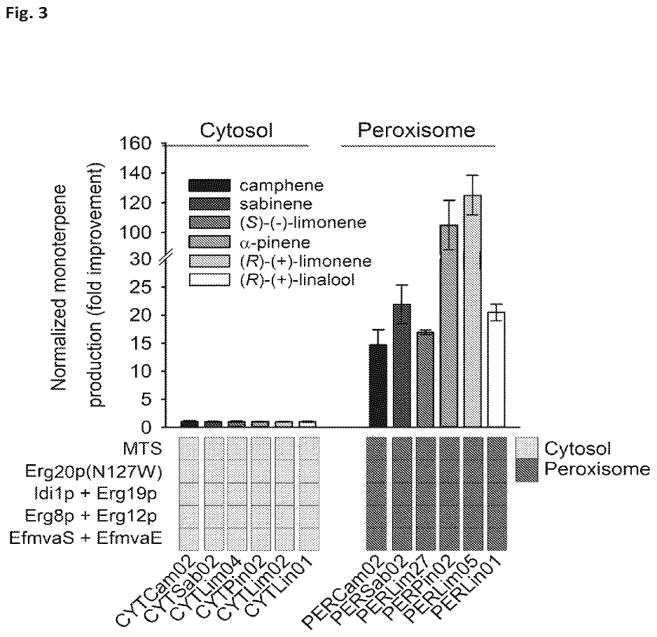

In order to assess whether the peroxisome-linked improvements reported in examples 1 and 2 were specific to limonene production or were applicable to monoterpenes in general, we targeted five additional MTPs either to the cytosol or to the peroxisome, along with Erg20p^{N127W}, with overexpression of the rest of the MVA pathway. A camphene synthase (SeCamS), a(S)-(−)-limonene synthase (MsLimS), a (R)-(+)-limonene synthase (C/LimS), a (R)-(+)-linalool synthase (McLiS), an alpha-pinene synthase (PtPinS), and a sabinene synthase (SpSabS), were chosen and evaluated by determining the production titer of their major product. The same positive effect of peroxisome targeting of these five monoterpene synthase (MTSs) together with a GPP synthase (Erg20p^{N127W}) was observed with an improvement of 14-fold (PERCam02 vs CYTCam02), 17-fold (PERPin02 vs CYTPin02), 22-fold (PERSab02 vs CYTSab02), 17-fold (PERLim27 vs CYTLim04), 125-fold (PERLim05 vs CYTLim02) and 20.5-fold (PERLin01 vs CYTLin01), for camphene, pinene, sabinene, (S)-(−)-limonene, (R)-(+)-linonene and (R)-(+)-linalool respectively, compared to the corresponding cytosolic expression of the same enzymes (FIG. 3).

### Example 4: Improved Monoterpene Production Using an Optimized Buffered Synthetic Minimal Media

A synthetic minimal defined media was used in order to assess production in an industrially relevant media. This synthetic minimal defined media was composed of the following: 5 g/L (NH₄)₂SO₄; 3 g/L KH₂PO₄; 1 g/L MgSO₄*7 H₂O; 0.0064 g/L D-biotin; 0.03 g/L nicotinic acid; 0.1 g/L thiamin HCL; 0.04 g/L D-panthothenic acid; 0.08 g/L myo-inositol; 0.02 g/L pyridoxine; 0.067 g/L tritriplex III; 0.067 g/L (NH₄)₂Fe(SO₄)₂·6H₂O; 0.0055 g/L CuSO₄; 0.02 g/L ZnSO₄; 0.02 g/L MnSO₄; 0.00125 g/L NiSO₄; 0.00125 g/L CoCL₂; 0.00125 g/L boric acid; 0.00125 g/L KI and 0.00115 Na₂MoO₄. The pH is hereby buffered with MES at a starting value of 6.3.

Strains PERLim05, PERGer02, PERPin02 and PERLin01 were used to determine monoterpene production levels in this medium. Additionally, a strain named PERMyr01, producing beta-myrcene, was constructed by introducing the beta-myrcene synthase ObMyrS together with the GPP synthase ERG20N127 and the rest of the MVA pathway targeted to the peroxisome by fusion with the C-terminal tri-peptide SKL (SEQ ID NO: 26). All strain were cultivated in shake flask for 72 h at 30 degrees with 10% isopropyl myristate overlay. The synthetic minimal defined medium described previously was used and supplemented with 4% galactose for growth and gene inductions.

Strain PERLim05 produces 770 mg of limonene/L of culture, strain PERGer02 produces 1681 mg of geraniol/L of culture, strain PERPin02 produces 250 mg of alpha-pinene/L of culture, strain PERLin01 produces 547 mg of linalool/L of culture and strain PERMyr01 produces 251 mg of myrcene/L of culture. This represents a 5.4-fold, 5.2-fold, 5.1-fold and 2.9-fold improvement for strains PERLim05, PERGer02, PERPin02 and PERLin01 respectively, compared to the same strains cultivated in the un-buffered complete media used in example 3.

### Example 5: High Levels of (+)-Limonene and Geraniol Production by a Combined Strategy of Genomic Integration and Plasmid-Based Expression of the MVA Pathway Genes Together with a GPPS and LimS or GES

A single copy of each gene of the MVA pathway targeted to the peroxisome was integrated into the genome of strain EGY48 together with ERG20p^{N127W} and C/LimS/tObGES giving strain PERLim06 and strain PERGer01. Furthermore, an additional copy of each gene of the MVA pathway, Erg20p^{N127W} and C/LimS or tObGES were introduced on plasmids in strains PERLim06 and strain PERGer01 giving strains PERLim07 and PERGer02, respectively.

In order to determine the maximum limonene and geraniol titer achievable by compartmentalizing the pathway into the peroxisome, semi-continuous fed-batch experiment were carried out with strains PERLim07 and PERGer02. The cultures were fed every 48 h with 40 g/L galactose and 20 g/L raffinose and the pH was adjusted to 4.5. The IPM layer was also harvested every 48 h to measure monoterpenes production.

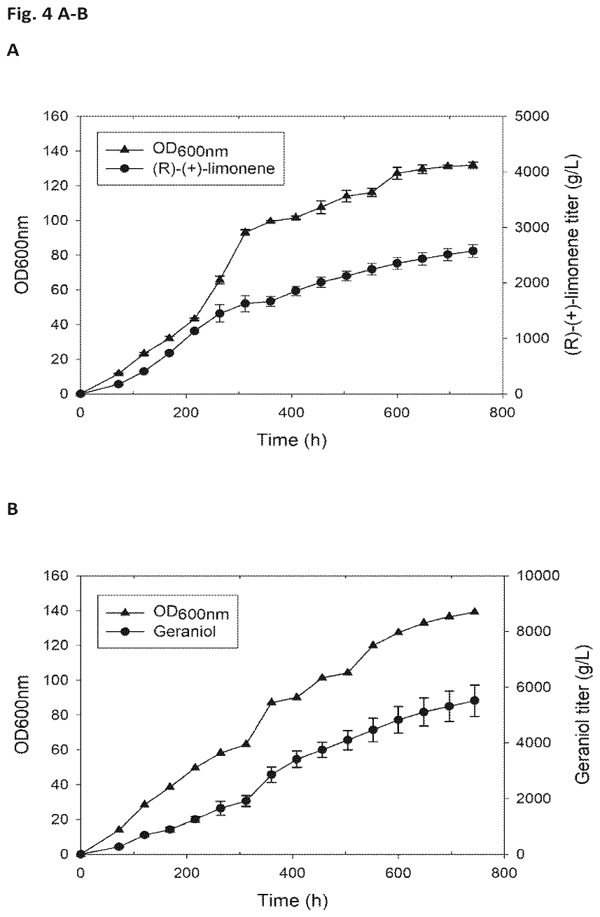

Fed batch flask culture with strains PERLim07 and PERGer02 resulted on a continuous accumulation of limonene and geraniol that was proportional, to a great extent, to the amount of biomass formed. After 700 h, titers of 2575 mg of limonene/L of culture (FIG. 4A) and 5516 mg of geraniol/L of culture (FIG. 4B) were determined.

### Example 6: Peroxisomal Localization of a Fusion Protein Comprising of a GPP Synthase Domain and a Terpene Synthase Domain Increases Terpenes Production

The possibility of using a single polypeptide having both a GPP synthase and a terpene synthase activity is investigated in this example of the present invention. Such a bifunctional enzyme could be already found in nature or synthetically created. For exemplification purpose, we created such a polypeptide by fusing a GPP synthase domain and a terpene synthase domain together and targeted it to the peroxisome.

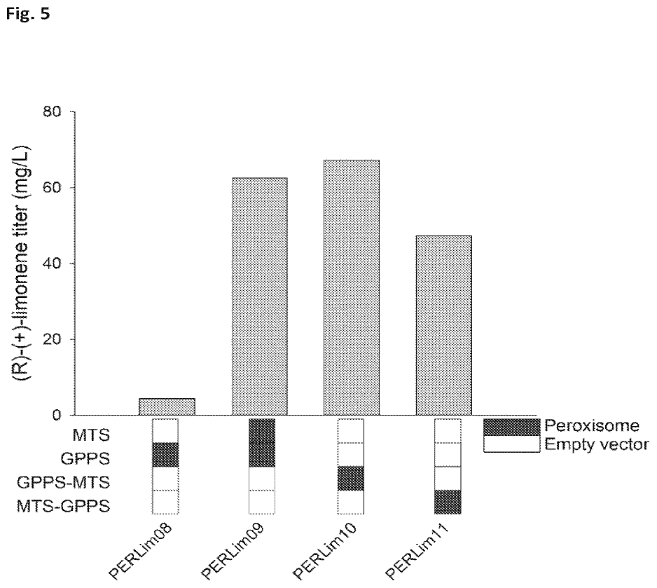

To do so, the GPP synthase Erg20p^{N127W} was fused to the terpene synthase C/LimS by a linker polypeptide comprised of five glycine-serine repeats (5×GS). The GPP synthase domain can be at the N-terminal end of the protein and the terpene synthase domain at the C-terminal end of the protein. Alternatively, the terpene synthase domain can be at the N-terminal end of the protein and the GPP synthase domaine at the C-terminal end of the protein. In this example, both these configurations were tested. Construction of those two synthetic enzymes gave two new polypeptides described in SEQ ID NO 15 and SEQ ID NO 16. Both sequences were subsequently introduced into an expression vector for yeast to give plasmids pPER15 and pPER16 and the strain PERLim06 was transformed with either one of the two plasmids to give PERLim10 and PERLim11. After culturing PERLim10 and PERlim11 for 72 h at 30° C. with an IPM overlay, limonene production was measured and compared to the one of strains PERLim08 and PERlim09. As seen in FIG. 5, limonene production obtained by peroxisomally targeting one or the other bifuctional GPP-terpene synthase fusion (PERLim10 and PERLim11) is similar or better compared to the production observed when peroxisomally targeting a GPP synthase and a terpene synthase as separate enzyme (PERLim09). Furthermore, a 16-fold and an 11-fold improvement in limonene production are observed when the bifuctional GPP-terpene synthase is targeted to the peroxisome compared with targeting only the GPP synthase activity to the peroxisome (PERLim08).

### Example 7A: Efficient Production of Trans-Isopiperitenol, Precursor of Menthol

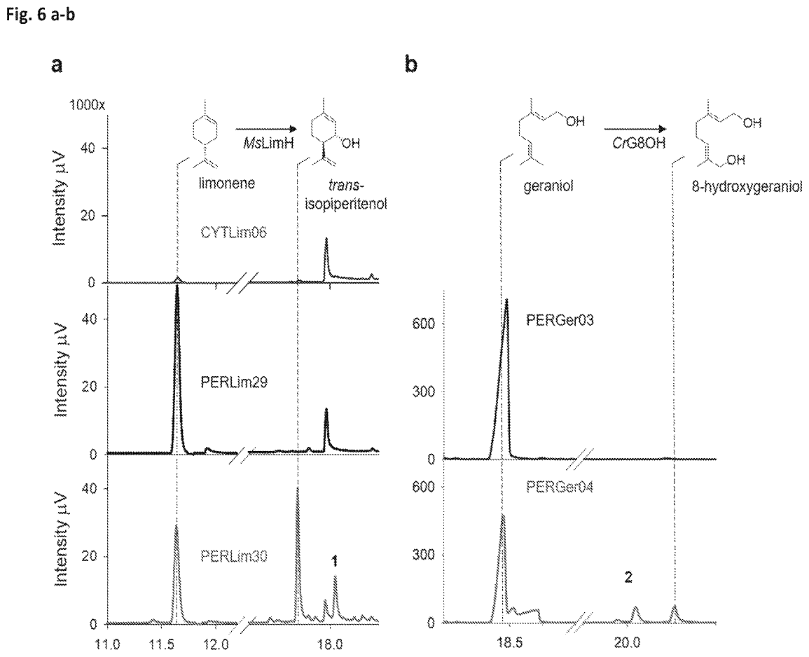

We further evaluated the contribution of the present invention in the production of trans-isopiperitenol, which is the precursor of the high-value compound menthol. We introduced the limonene-3-hydroxylase from Mentha spicata (MsLim3H; Q6IV13.1) into strain PERLim27, together with the cytochrome P450 reductase (tcCPR/POR) from Taxus cuspidata to give strain PERLim30 or an empty vector (pESC-Leu) to give PERLim29. As a comparison we introduced the limonene-3-hydroxylase from Mentha spicata (MsLim3H; Q6IV13.1) into strain CYTLim04, together with the cytochrome P450 reductase (tcCPR/POR) from Taxus cuspidata to give strain CYTLim06. After 72 h of growth in complete minimal media, production of trans-isopiperitenol was evaluated by GC-FID of the culture extracts. As shown in FIG. 6A, both (−)-limonene and trans-isopiperitenol could be extracted from the strain PERLim30, while only limonene was detected in strain PERLim29 lacking the limonene-3-hydroxylase MsLim3H and the cytochrome P450 reductase tcCPR (FIG. 6A). Reduction of the (−)-limonene recovered from the PERLim30 culture compared to PERLim29 is in accordance to a 37% conversion into trans-isopiperitenol (19.24 mg/L) (FIG. 6A). However when limonene production was done in the cytosol in strain CYTLim06, only 0.28 mg/L trans-isopiperitenol was obtained, corresponding to a limonene conversion of only 14%. These results demonstrate that increased limonene production in the peroxisome allow for a significant amount of it being channeled through the endoplasmic reticulum (ER), where it can be hydroxylated by MsLim3H.

### Example 7B: Efficient Production of 8-Hydroxygeraniol, Precursor of Iridoids and Monoterpene Indole Alkaloids

We further evaluated the contribution of the present invention in the production of 8-hydroxygeraniol, which is the precursor of large groups of high-value compounds, including the iridoids and the monoterpene indole alkaloids. We introduced the geraniol 8-hydroxylase from Catharanthous roseus (CrG8OH; CYP76B6) into strain PERGer02, together with the cytochrome P450 reductase (CrCPR/POR) from the same species to give strain PERGer04 or an empty vector (pESC-Leu) to give PERGer03. After 72 h of growth in complete minimal media, production of 8-hydroxy-geraniol was evaluated by GC-FID of the culture extracts. As shown in FIG. 6B, both geraniol and 8-hydroxygeraniol could be extracted from the strain PERGer04, while only geraniol was detected in strain PERGer03 lacking the geraniol 8-hydroxylase CrG8OH and the cytochrome P450 reductase CrCPR (FIG. 6B). Reduction of the geraniol recovered from the PERGer04 culture compared to PERGer03 is in accordance to a partial conversion into 8-hydroxy-geraniol (FIG. 6B). These results demonstrate that increased geraniol production in the peroxisome allow for a significant amount of it being channeled through the endoplasmic reticulum (ER), where it can be hydroxylated by CrG8OH.

### Example 8: Efficient Production of Cannabinoids by Targeting a GPP Synthase and a Geranyldiphosphate:Olivetolate Geranyltransferase to the Peroxisome

The invention was evaluated for its applicability in the production of another group of GPP-derived high-value compounds, beyond monoterpenoids and monoterpene indole alkaloids, that of cannabinoids. In the cannabinoid biosynthetic pathway, olivetolic acid (OA) is prenylated by GPP to form cannabigerolic acid (CBGA) via the action of a dedicated geranyltransferase. CBGA represents a key step in the pathway because it is the last common precursor to various types of cannabinoids, such as tetrahydrocannabinolic acid (THCA) and cannabidiolic acid (CBDA). In yeast, this prenylation step represents a major bottleneck in the process of producing high titers of cannabinoids because of the limited pool of GPP in the cytosol.

In this example, the GPP synthase Erg20p^{N127W} and the geranyldiphosphate:olivetolate geranyltransferase CsPT4, from C. sativa, were targeted to the yeast peroxisome, using the C-terminal targeting signal SKL, for CBGA production. Both genes were introduced into the strain PERMva01 under the control of the inducible promoters P_{GAL1} and P_{GAL10}, to give strain PERCan01. After 72 h of growth in complete minimal media under galactose-induced conditions and supplemented with various concentration of olivetolic acid (0.05 mM, 0.1 mM, 0.25 mM or 0.5 mM), production of CBGA was analyzed by LC-MS. Cells were disrupted and CBGA extracted from the cell fraction (pellet) using ethyl acetate/formic acid (0.05% v/v) in a 1:1 ratio and glass bead beating. The organic layer was separated by centrifugation and evaporated using a SpinVac. The remaining dry fraction was dissolved in methanol and filtered through a 0.22 μm pore size PVDF filter. Samples were diluted 10 times prior to LC-MS analysis.

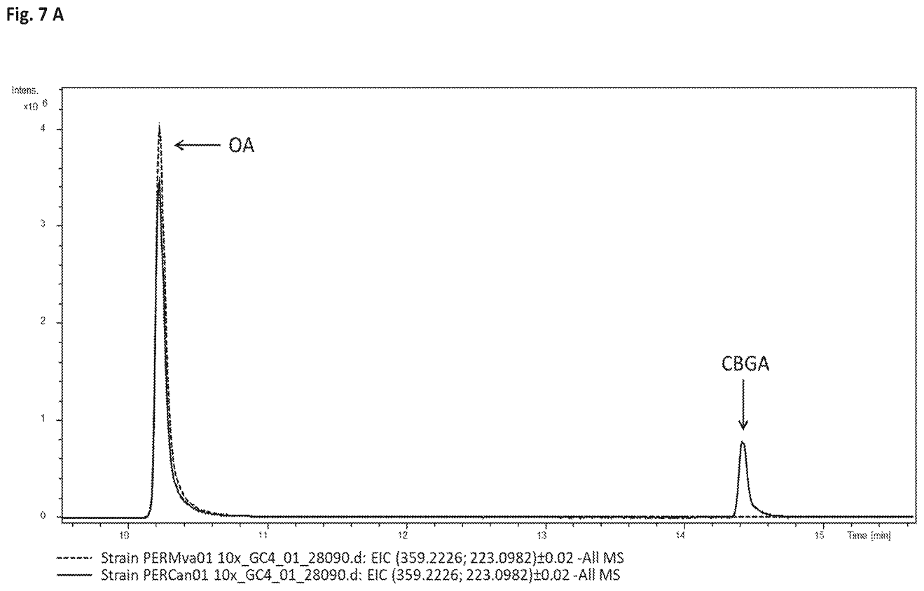

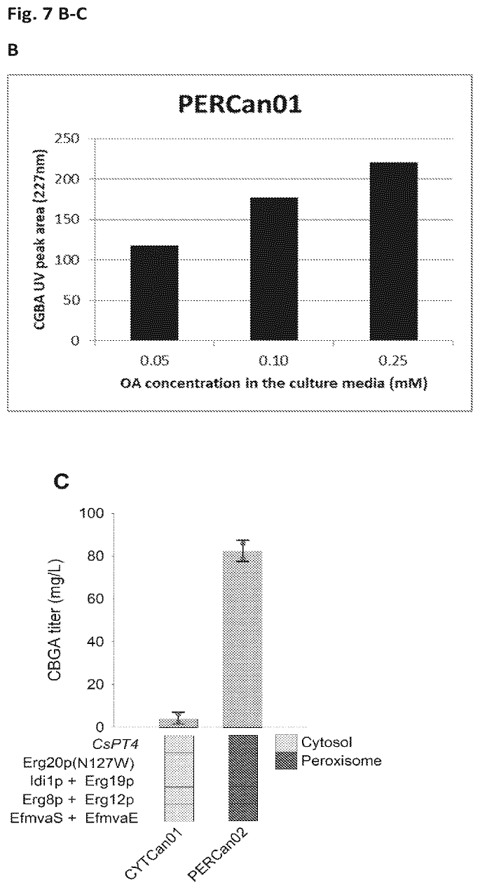

As shown in FIG. 7A, both OA and CBGA could be extracted from the strain PERCan01, while only OA was detected in strain PERMva01 lacking the GPP synthase ERG20p^{N127W} and the geranyldiphosphate:olivetolate geranyltransferase CsPT4. Reduction of the OA recovered inside the cells in PERCan01 compared to PERMva01 is in accordance to a partial conversion into CBGA. These results demonstrate that 1) OA can be transported and/or diffuse into the peroxisome, 2) CsPT4 is active in the peroxisome and 3) the pool of GPP is sufficient in the peroxisome to allow efficient OA prenylation.

Additionally, an alternative N-terminal peroxisomal targeting signal was examined for CsPT4 (SEQ ID NO: 29). PTS2-CsPT4 was introduced in strain PERMva01 together with the GPP synthase Erg20p^{N127W}-SKL giving both enzymes peroxisomal localization. This new strain called PERCan02 was assessed as previously for OA consumption and CBGA production. For comparison, CsPT4 and Erg20p^{N127W} were introduced in strain CYTMva01 to give strain CYTCan01 for cytosolic CBGA production. As shown in FIG. 7B, strain PERCan02 produced 82.3 mg/L CBGA, 19.5-fold more than strain CYTCan01 with only 4.2 mg/L CBGA.

### REFERENCES

Ellerstrom M et al (1992): Cloning of a cDNA for rape chloroplast 3-isopropylmalate dehydrogenase by genetic complementation in yeast. Plant Mol Biol, 18, 557-560.
Ignea C. et al (2011): Improving yeast strains using recyclable integration cassettes for the production of plant terpoenoids. Microb. Cell. Fact, 10, 4 (3)
Ignea C. et al (2014): Engineering monoterpene production in yeast using a synthetic dominant negative geranyl diphosphate synthase. ACS Synth. Biol. 3, 298-306. (53)
Ignea C. et al (2018): Synthesis of 11-carbon terpenoids in yeast using protein and metabolic engineering. Nature Chemical Biology 14, 1090-1098.
Nour-Eldin H. H et al (2010): USER cloning and USER fusion: The Ideal cloning techniques for small and big laboratories. In Fett-Neto A (eds.) Plant Secondaty Metabolism Engineering. Methods in Molecular biology (Methods and Protocols), vil 643. Humana Press, Totowa, NJ.
Orlova I et al. (2009): The Small Subunit of Snapdragon Geranyl Diphosphate Synthase Modifies the Chain Length Specificity of Tobacco Geranylgeranyl Diphosphate Synthase in Planta. Plant Cell; 21, 4002-4017.
Philips M. A. et al (2003): cDNA isolation, functional expression, and characterization of (+)-α-pinene synthase and (−)-α-pinene synthase form lobolly pine (Pinus taeda): Stereocontrol in pinene biosynthesis. Arch. Biochem. Biophys. 411, 267-276. (57)
Thomas B. J. and R. Rothstein (1989): Elevated recombination rates in transcriptionally active DNA. Cell 56, 619-630 (4)

1. A recombinant yeast cell, comprising a peroxisomally-localized GPP synthase and a peroxisomally-localized monoterpene synthase.

2. The recombinant yeast cell of claim 1, wherein the yeast cell belongs to one of the genera: Saccharomyces, Pichia, Candida, Ogataea, or Yarrowia.

3. The recombinant yeast cell of claim 2, wherein the yeast cell is selected from the species: Saccharomyces cerevisiae, Pichia pastoris, Candida albicans, Candida boidinii, Ogataea polymorpha, and Yarrowia lipolytica.

4. The recombinant yeast cell of claim 1, wherein the yeast cell is a Saccharomyces cerevisiae cell.

5. The recombinant yeast cell of claim 1, wherein peroxisomal localization is provided by a peroxisomal localization signal.

6. The recombinant yeast cell of claim 5, wherein the peroxisomal localization signal is SKL (SerLysLeu) added to the C-terminus of the GPP synthase and/or the monoterpene synthase, or a C-terminal tripeptide having sequence (S/A/C)(K/R/H)-(L/M).

7. The recombinant yeast cell of claim 5, wherein the peroxisomal localization signal comprises an N-terminal peptide having sequence (R/K)-(L/V/I)-X5-(H/Q)-(L/A/F).

8. The recombinant yeast cell of claim 1, wherein the GPP synthase is Erg20p^{N127W}.

9. The recombinant yeast cell of claim 1, wherein the GPP synthase is an engineered GPP synthase comprising a mutation that prevents further conversion of GPP into FPP by addition of an IPP molecule.

10. The recombinant yeast cell of claim 1, wherein the monoterpene synthase is selected from the group consisting of (+)-limonene synthase, (−)-limonene synthase, alpha-pinene synthase, sabinene synthase, camphene synthase, geraniol synthase, linalool synthase, and myrcene synthase.

11. The recombinant yeast cell of claim 10, wherein the monoterpene synthase is selected from the group consisting of geraniol synthase, linalool synthase, and myrcene synthase.

12. The recombinant yeast cell of claim 1, further comprising peroxisomally-localized EfmvaS, EfmvaE, ERG12, ERG8, ERG19, and IDI.

13. The recombinant yeast cell of claim 1, wherein the GPP synthase and the monoterpene synthase are present as a single fusion protein comprising a GPP synthase domain and a monoterpene synthase domain.

14. The recombinant yeast cell of claim 13, wherein the fusion protein comprises Erg20p^{N127W} and a limonene synthase linked by a 5×GS polypeptide and targeted to the peroxisome by a C-terminal PTS1.

15. The recombinant yeast cell of claim 4, wherein the monoterpene synthase is geraniol synthase.

16. The recombinant yeast cell of claim 4, wherein the monoterpene synthase is linalool synthase.

17. The recombinant yeast cell of claim 4, wherein the monoterpene synthase is myrcene synthase.
